(19) United States
(12) Patent Application Publication
(10) Pub. No.: US 2024/0270726 A1
(43) Pub. Date: Aug. 15, 2024

(54) A NOVEL PROCESS FOR THE PREPARATION OF ANTHRANILIC DIAMIDES

(71) Applicant: PI INDUSTRIES LIMITED, Udaipur -Rajasthan (IN)

(72) Inventors: Ajay YADAV, Udaipur, Rajasthan (IN); Tridib MAHAPATRA, Udaipur, Rajasthan (IN); Jagadish PABBA, Udaipur, Rajasthan (IN); Pranab KUMAR PATRA, Udaipur Rajasthan (IN); Bharat Uttamrao SHINDE, Ahmednagar, Maharashtra (IN); Amol D. KALWAGHE, Ahmednagar, Maharashtra (IN); Jigarkumar Harikishandas SHAH, Maharashtra (IN); Shrikant Bhausaheb KANAWADE, Nashik, Maharashtra (IN); Raju SHARMA, Hoshiarpur, Punjab (IN); Bhagwan Lal GURJAR, Udaipur, Rajasthan (IN); Alexander G.M. KLAUSENER, Pulheim (DE)

(73) Assignee: PI INDUSTRIES LTD., Udaipur-Rajasthan (IN)

(21) Appl. No.: 18/565,739

(22) PCT Filed: Jun. 3, 2022
(86) PCT No.: PCT/IB2022/055195

§ 371 Date: Nov. 30, 2023

(30) Foreign Application Priority Data
Jun. 4, 2021 (IN) 202111024851

(51) Int. Cl. C07D 409/14

(52) U.S. Cl. CPC C07D 409/14

(57) ABSTRACT

The present invention relates a novel process for preparing compounds of formula (I) or salts or N-oxides thereof,

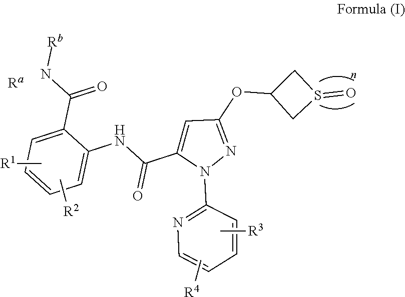

wherein,
R^{a}, R^{b}, R¹, R², R³, R⁴ and n are as defined in the description.

The process for preparing a compound of formula (I) comprises step of: reacting a compound of formula (IV) with substituted anthranilic acid of formula (III) and suitable amine of formula (R^{a}R^{b}NH) or with substituted anthranilic amide of formula (IIIa), optionally isolating compound of formula (II).

Further, the present invention also describes the preparation of compound of formula (IV) which is obtained from compound of formula (VIII).

### FIELD OF THE INVENTION

The present invention relates to a process for the preparation of anthranilic diamides of formula (I) and their intermediate compounds of formula (IV) or salts or N-oxides thereof,

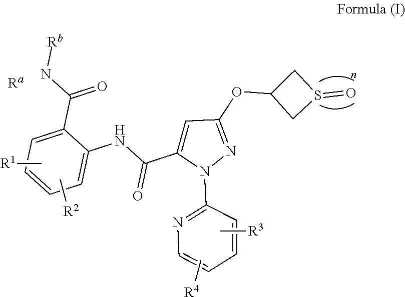

wherein, n, R¹, R^{a}, R^{b}, R², R³ and R⁴ are as defined in the description.

The present invention further relates to a process for the preparation of compounds of formula (IV)

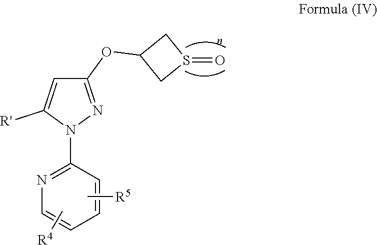

wherein n, R′, R³ and R⁴ are as defined in the description.

### BACKGROUND OF THE INVENTION AND PROBLEM TO BE SOLVED

1-Pyridinylpyrazole-5-carboxylic acids are known to be important intermediates in the agrochemical industry, e. g. for the synthesis of anthranilic diamides which are useful to protect crops against harmful pests. Several methods have been disclosed in the literature, by which these intermediates can be obtained.

WO2019150220 has described novel anthranilic diamides, and their use as insecticide

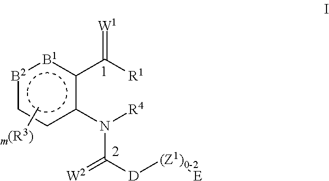

wherein, D represents

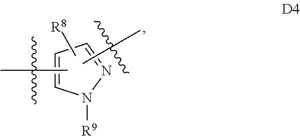

Z¹ is independently a direct bond or CR⁶R⁷ or NR^{c} or O or S(O)₀₋₂; and E represents 4 membered heterocycles.

The prior art has also described processes for preparing the said compounds of formula (I). However, the processes described in the prior art have shortcomings such as poor yields of the desired intermediates or products, or synthetic procedures being not amenable to commercial scale, or of involving extreme reaction conditions making them uneconomical.

Thus, there is a need for a process that obviates at least one of the shortcomings associated with the known processes.

The present invention provides a process for the preparation of compounds of formula (I) (thietanyloxy anthranilic diamide) which provides a good yield on a commercial scale.

The present invention also provides a process for the preparation of compounds of formula (IV) (thietanyloxy pyrazolopyridine) which addresses at least one of the shortcomings mentioned in the prior art.

### OBJECTIVE OF THE INVENTION

It is therefore an objective of the present invention to provide a novel and economically viable process for the preparation of anthranilic diamides of formula (I).

Another objective of the present invention is to provide novel compounds of formula (IV) which are used for the preparation of such anthranilic diamides of formula (I).

Yet another objective of the present invention is to provide a process for the preparation of compounds of formula (IV).

The present invention provides a solution to these objectives by offering a novel high yielding and economical process that allows for the preparation of anthranilic diamides and/or novel key intermediates to prepare such anthranilic diamides, overcoming at least one of the shortcomings of the processes described in the prior art.

### SUMMARY OF THE INVENTION

These objectives were achieved according to the present invention by providing a novel process for preparing the anthranilic diamides of formula (I),

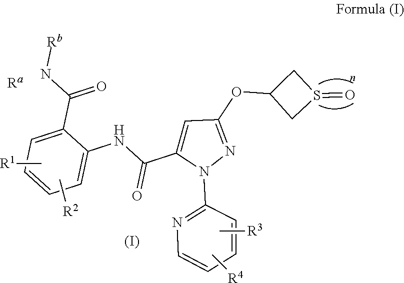

wherein,
R^{a} and R^{b} are independently selected from the group consisting of hydrogen, C₁-C₆ alkyl, C₃-C₆ cycloalkyl and C₃-C₆ cycloalkyl-C₁-C₄ alkyl; wherein R^{a} and R^{b} are optionally substituted with one or more halogen;
R¹ and R² are independently selected from the group consisting of hydrogen, halogen, cyano, C₁-C₆ alkyl, C₁-C₄ haloalkyl and C₃-C₆ cycloalkyl;
R³ is selected from the group consisting of halogen, C₁-C₄ alkyl and C₁-C₄ haloalkyl;
R⁴ is selected from the group consisting of hydrogen, halogen, C₁-C₄ alkyl and C₁-C₄ haloalkyl; n represents an integer selected from 0 to 2; or salts or N-oxides thereof comprising the steps of:
A. converting a compound of formula (V) to a compound of formula (IV);

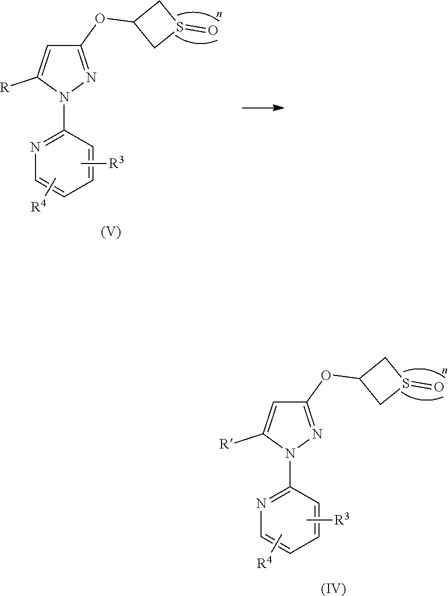

wherein, R is selected from CX₃, CN or COORS, Re represents C₁-C₄ alkyl, R′ represent COOH or COX, X is halogen, n, R³ and R⁴ are as defined herein above;
B. reacting the compound of formula (IV) with a compound of formula (III) and a suitable amine of formula (R^{a}R^{b}NH) or with a compound of formula (IIIa) to obtain a compound of formula (I);

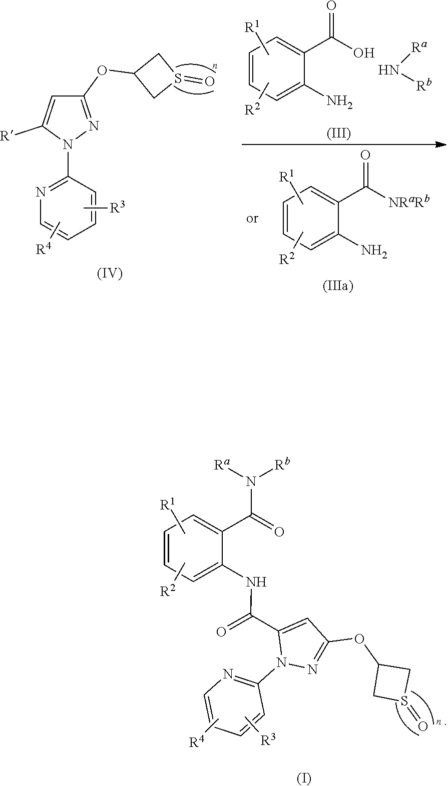

wherein, n, R′, R^{a}, R^{b}, R¹, R², R³ and R⁴ are as defined herein above.

In another embodiment, the compound of formula (IV) is obtained from the compound of formula (VIII) as shown in scheme below,

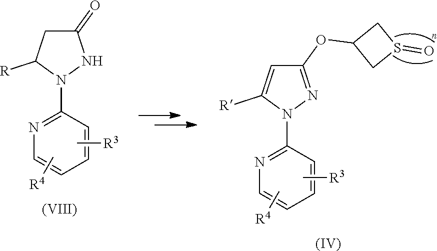

wherein, n, R, R′, R³ and R⁴ are as defined herein above.

### DETAILED DESCRIPTION OF THE INVENTION

### General Definitions

The definitions provided herein for the terminologies used in the present disclosure are for illustrative purpose only and in no manner limit the scope of the present invention disclosed in the present disclosure.

As used herein, the terms “comprises”, “comprising”, “includes”, “including”, “has”, “having”, “contains”, “containing”, “characterized by” or any other variation thereof, are intended to cover a non-exclusive inclusion, subject to any limitation explicitly indicated. For example, a composition, mixture, process or method that comprises a list of elements is not necessarily limited to only those elements but may include other elements not expressly listed or inherent to such composition, mixture, process or method.

The transitional phrase “consisting of” excludes any element, step or ingredient not specified. If in the claim, such would close the claim to the inclusion of materials other than those recited except for impurities ordinarily associated therewith. When the phrase “consisting of” appears in a clause of the body of a claim, rather than immediately following the preamble, it limits only the element set forth in that clause; other elements are not excluded from the claim as a whole.

The transitional phrase “consisting essentially of” is used to define a composition or method that includes materials, steps, features, components or elements, in addition to those literally disclosed, provided that these additional materials, steps, features, components or elements do not materially affect the basic and novel characteristic(s) of the claimed invention. The term “consisting essentially of” occupies a middle ground between “comprising” and “consisting of”.

Further, unless expressly stated to the contrary, “or” refers to an inclusive “or” and not to an exclusive “or”. For example, a condition A “or” B is satisfied by any one of the following: A is true (or present) and B is false (or not present), A is false (or not present) and B is true (or present), and both A and B are true (or present).

Also, the indefinite articles “a” and “an” preceding an element or component of the present invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

Carbon-based radical refers to a monovalent molecular component comprising a carbon atom that connects the radical to the remainder of the chemical structure through a single bond. Carbon-based radicals can optionally comprise saturated, unsaturated and aromatic groups, chains, rings and ring systems, and heteroatoms. Although carbon-based radicals are not subject to any particular limit in size, in the context of the present invention they typically comprise 1 to 16 carbon atoms and o to 3 heteroatoms. The carbon-based radicals are selected from C₁-C₆ alkyl, C₁-C₆ haloalkyl and phenyl optionally substituted with 1-3 substituents selected from C₁-C₃ alkyl, halogen and nitro.

The meaning of various terms used in the description shall now be illustrated.

The term “alkyl”, used either alone or in compound words such as “alkylthio” or “haloalkyl” or —N(alkyl) or alkylcarbonylalkyl or alkylsuphonylamino includes straight-chain or branched C₁ to C₁₀ alkyl, preferably C₁ to C₆ alkyl, more preferably C₁ to C₄ alkyl. Non-limiting examples of alkyl include methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl or the different isomers. If the alkyl is at the end of a composite substituent, as, for example, in alkylcycloalkyl, the part of the composite substituent at the start, for example the cycloalkyl, may be mono- or polysubstituted identically or differently and independently by alkyl. The same also applies to composite substituents in which other radicals, for example alkenyl, alkynyl, hydroxyl, halogen, carbonyl, carbonyloxy and the like, are at the end.

The term “cycloalkyl” means alkyl closed to form a ring. Non-limiting examples include cyclopropyl, cyclobutyl, cyclopentyl and cyclohexyl. This definition also applies to cycloalkyl as a part of a composite substituent, for example cycloalkylalkyl etc., unless specifically defined elsewhere.

The term “halogen”, either alone or in compound words such as “haloalkyl”, includes F, C₁, Br or I. Further, when used in compound words such as “haloalkyl”, said alkyl may be partially or fully substituted with halogen atoms which may be the same or different.

Non-limiting examples of “haloalkyl” include chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl, 1,1-dichloro-2,2,2-trifluoroethyl, and 1,1,1-trifluoroprop-2-yl. This definition also applies to haloalkyl as a part of a composite substituent, for example haloalkylaminoalkyl etc., unless specifically defined elsewhere.

Hydroxy means —OH, Amino means —NRR, wherein R can be H or any possible substituent such as alkyl. Carbonyl means —C(O)—.

Halocycloalkyl, halocycloalkenyl, alkylcycloalkyl, cycloalkylalkyl, cycloalkoxyalkyl, alkylsulfinylalkyl, alkylsulfonylalkyl, haloalkylcarbonyl, cycloalkylcarbonyl, haloalkoxylalkyl, and the like, are defined analogously to the above examples.

“Alkylamino”, “dialkylamino”, and the like, are defined analogously to the above examples.

The total number of carbon atoms in a substituent group is indicated by the “Cᵢ-Cⱼ” prefix where i and j are numbers from 1 to 21. For example, C₁-C₃ alkylsulfonyl designates methylsulfonyl through propylsulfonyl; C₂ alkoxyalkyl designates CH₃OCH₂; C₃ alkoxyalkyl designates, for example, CH₃CH(OCH₃), CH₃OCH₂CH₂ or CH₃CH₂OCH₂; and C₄ alkoxyalkyl designates the various isomers of an alkyl group substituted with an alkoxy group containing a total of four carbon atoms, examples including CH₃CH₂CH₂OCH₂ and CH₃CH₂OCH₂CH₂. In the above recitations, when a compound of formula (I) is comprised of one or more heterocyclic rings, all substituents are attached to these rings through any available carbon or nitrogen by replacement of a hydrogen on said carbon or nitrogen.

The term leaving group is a molecular fragment that departs with a pair of electrons in heterolytic bond cleavage. Leaving groups can be anions, cations or neutral molecules, but in either case it is crucial that the leaving group be able to stabilize the additional electron density that results from bond heterolysis. Common anionic leaving groups are halides such as Cl—, Br—, and I—, and sulfonate esters such as mesylates (MsO—), tosylate (TsO—) and triflate (CF₃SO₂O—).

When a compound is substituted with a substituent bearing a subscript that indicates the number of said substituents can exceed 1, said substituents (when they exceed 1) are independently selected from the group of defined substituents. Further, when the subscript m in (R)ₘ indicates an integer ranging from for example 0 to 4 then the number of substituents may be selected from the integers between 0 and 4 inclusive.

When a group contains a substituent which can be hydrogen, then, when this substituent is taken as hydrogen, it is recognized that said group is being un-substituted.

The embodiments herein and the various features and advantageous details thereof are explained with reference to the non-limiting embodiments in the description. Descriptions of well-known components and processing techniques are omitted so as to not unnecessarily obscure the embodiments herein. The examples used herein are intended merely to facilitate an understanding of ways in which the embodiments herein may be practiced and to further enable those of skilled in the art to practice the embodiments herein. Accordingly, the examples should not be construed as limiting the scope of the embodiments herein.

The description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the embodiments as described herein.

Any discussion of documents, acts, materials, devices, articles and the like that has been included in this specification is solely for the purpose of providing a context for the disclosure. It is not to be taken as an admission that any or all of these matters form a part of the prior art base or were common general knowledge in the field relevant to the disclosure as it existed anywhere before the priority date of this application.

The numerical values mentioned in the description and the description/claims though might form a critical part of the present invention of the present disclosure, any deviation from such numerical values shall still fall within the scope of the present disclosure if that deviation follows the same scientific principle as that of the present invention disclosed in the present disclosure.

The compounds synthesized by the novel and inventive process of the present invention may, if appropriate, be present as mixtures of different possible isomeric forms, especially of stereoisomers, for example E and Z, threo and erythro, and also optical isomers, but if appropriate also of tautomers. Both the E and the Z isomers, and also the threo and erythro isomers, and the optical isomers, any desired mixtures of these isomers and the possible tautomeric forms are disclosed and claimed.

In view of the above, the present invention provides a process for preparing anthranilic diamides of formula (I) or salts or N-oxides thereof,

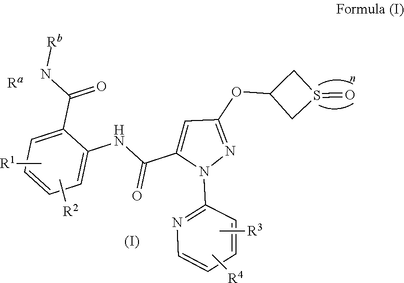

wherein,
R^{a} and R^{b} are independently selected from the group consisting of hydrogen, C₁-C₆ alkyl, C₃-C₆ cycloalkyl and C₃-C₆ cycloalkyl-C₁-C₄ alkyl; wherein R^{a} and R^{b} optionally substituted with one or more halogen;
R¹ and R² are independently selected from the group consisting of hydrogen, halogen, cyano, C₁-C₆ alkyl, C₁-C₄ haloalkyl, and C₃-C₆ cycloalkyl;
R³ is selected from the group consisting of halogen, C₁-C₄ alkyl and C₁-C₄ haloalkyl;
R⁴ is selected from the group consisting of hydrogen, halogen, C₁-C₄ alkyl and C₁-C₄ haloalkyl;
n represents an integer selected from 0 to 2;
   comprising the steps of:
A. converting a compound of formula (V) to a compound of formula (IV);

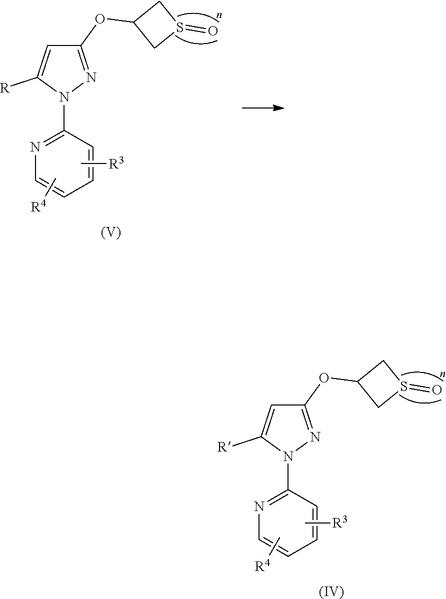

wherein, R is selected from CX₃, CN or COOR^{c}, R^{c} represents C₁-C₄ alkyl, R′ represent COOH or COX, X is halogen, R³ and R⁴ are as defined herein above;
B. reacting the compound of formula (IV) with a compound of formula (III) and a suitable amine of formula (R^{a}R^{b}NH) or with a compound of formula (III-a) to obtain a compound of formula (I);

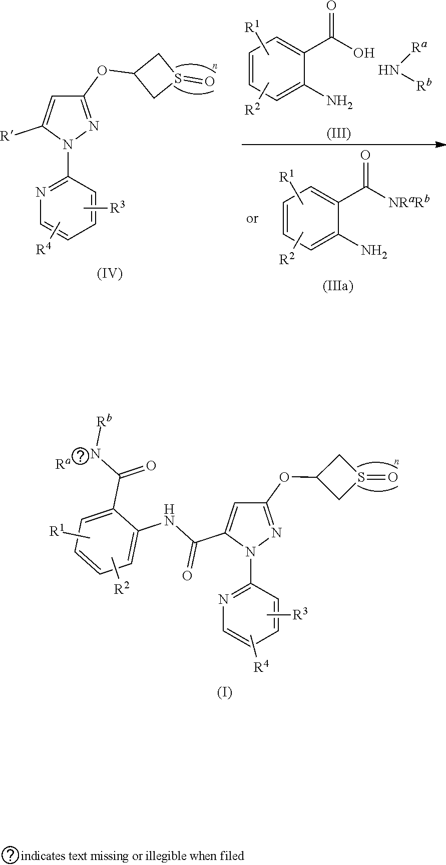

wherein, n, R′, R^{a}, R^{b}, R¹, R², R³ and R⁴ are as defined herein above.

The compounds of formula (III) or (IIIa) can be prepared by any of the processes as disclosed in the prior art.

In the above process, a compound of formula (IV) wherein R′ represents COOH, is reacted with a compound of formula (III) and a suitable amine of formula (R^{a}R^{b}NH), or with a compound of formula (IIIa) to obtain a compound of formula (I) in the presence of one or more suitable reagent(s) selected from mesyl chloride, thionyl chloride, tosyl chloride, cyanuric chloride and/or oxalyl chloride; preferably mesyl chloride.

In another embodiment, the present invention provides a process for preparing compounds of formula (IV) or salts thereof,

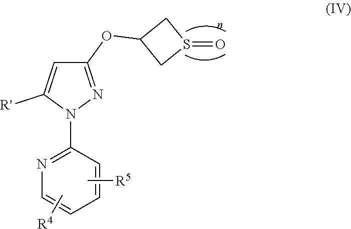

wherein,
R′ is selected from the group consisting of COOH and COX;
X represents halogen;
R³ is selected from the group consisting of halogen, C₁-C₄ alkyl and C₁-C₄ haloalkyl;
R⁴ is selected from the group consisting of hydrogen, halogen, C₁-C₄ alkyl and C₁-C₄ haloalkyl;
n represents an integer selected from 0 to 2;
   comprising the steps of:
a) obtaining a compound of formula (VII) from a compound of formula (VIII);

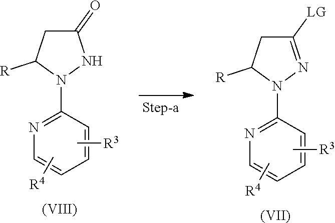

wherein, LG is selected from halogen, OMs, OTf, or OTs, R is selected from CX₃, CN or COOR^{c}, R^{c} represents C₁-C₄ alkyl, R³ and R⁴ are as defined herein above;
b) reacting the compound of formula (VII) with a compound of formula (X) to obtain a compound of formula (VI);

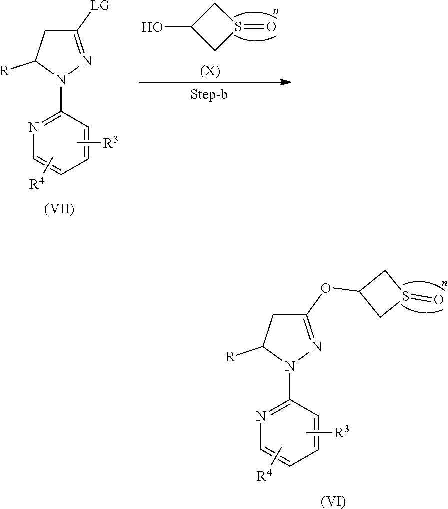

wherein, n=0-2, R, R³, R⁴ and LG are as defined herein above;
c) oxidizing the compound of formula (VI) with a suitable oxidizing agent and a suitable acid to obtain a compound of formula (V);

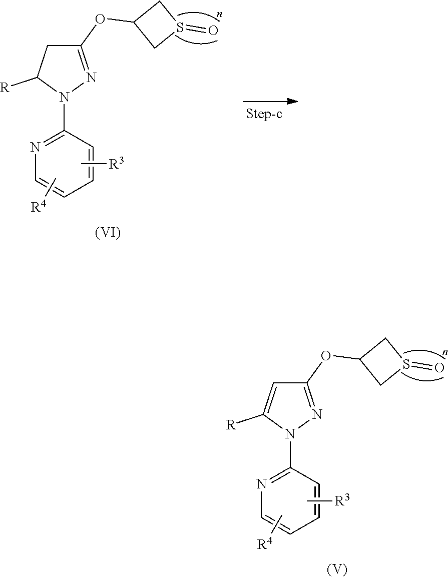

wherein, n, R, R³ and R⁴ are as defined herein above;
d) converting the compound of formula (V) to a compound of formula (IV);

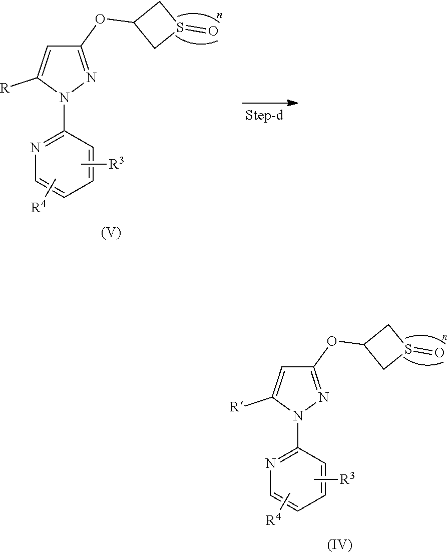

wherein, R′ represent COOH or COX, n, R, R³ and R⁴ are as defined herein above.

Alternatively, the compounds of formula (IV) can also be obtained by using the process comprising the steps of:
a) obtaining a compound of formula (IX) by oxidation of a compound of formula (VII);

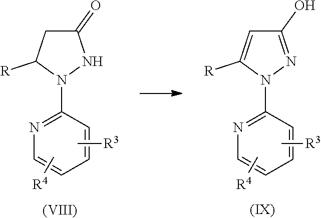

wherein, R is selected from CX₃, CN or COOR^{c}, R^{c} represents C₁-C₄ alkyl, R³ and R⁴ are as defined herein above;
b) converting the compound of formula (IX) to a compound of formula (V);

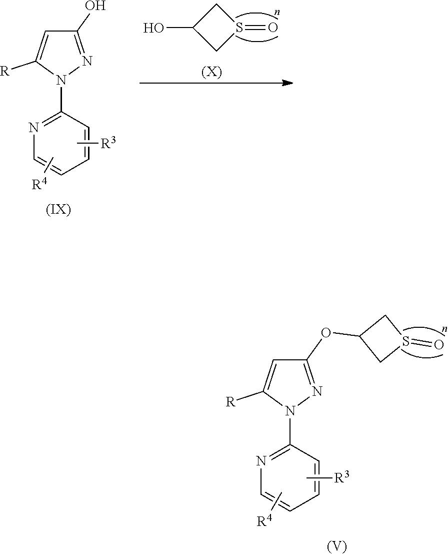

wherein, R, R³, R⁴ are as defined herein above;
c) converting the compound of formula (V) to a compound of formula (IV);

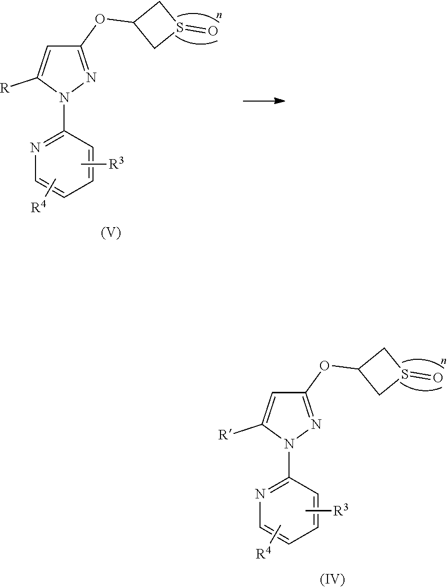

wherein, R′ represent COOH or COX, R, R³ and R⁴ are as defined herein above.

In one embodiment, the compound of formula (IV) wherein n=1 or 2 can be obtained from a compound of formula (VI),

comprising the steps of:
b) obtaining a compound of formula (V) by oxidation of a compound of formula (VI) using a suitable oxidizing agent;

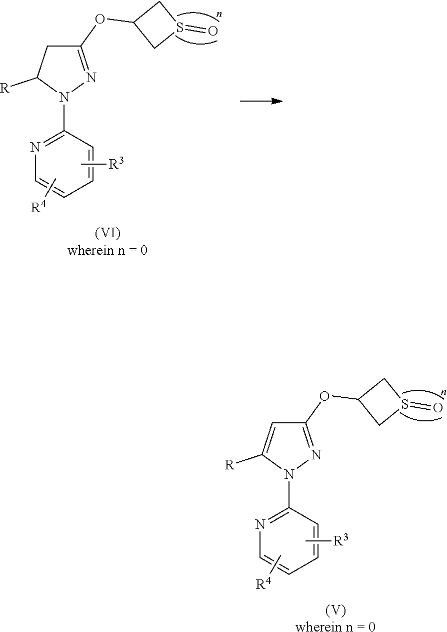

wherein, R is selected from CX₃, CN or COOR^{c}, R^{c} represents C₁-C₄ alkyl, R³ and R⁴ are as defined herein above;
c) converting the compound of formula (V) to a compound of formula (V) using a suitable oxidizing agent;

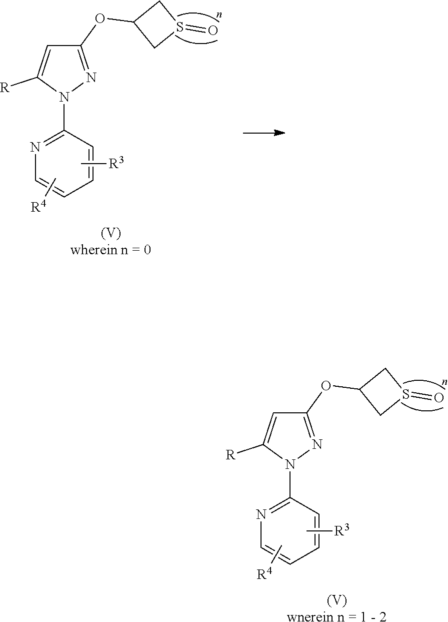

wherein, R, R³, R⁴ are as defined herein above;
d) converting the compound of formula (V) to a compound of formula (IV);

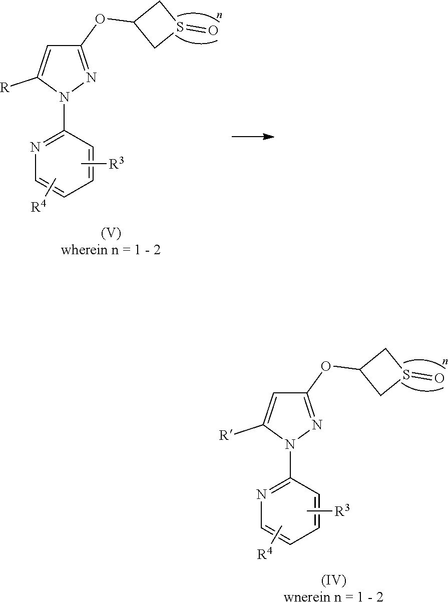

wherein, R′ represent COOH or COX, R, R³ and R⁴ are as defined herein above.

In one embodiment, the compound of formula (V) wherein n=1 or 2 can be obtained from a compound of formula (VI), comprising the steps of:
c) converting a compound of formula (VI) to a compound of formula (VI), using a suitable oxidizing agent, which on further reaction with a suitable acid to obtain compound of formula (V);

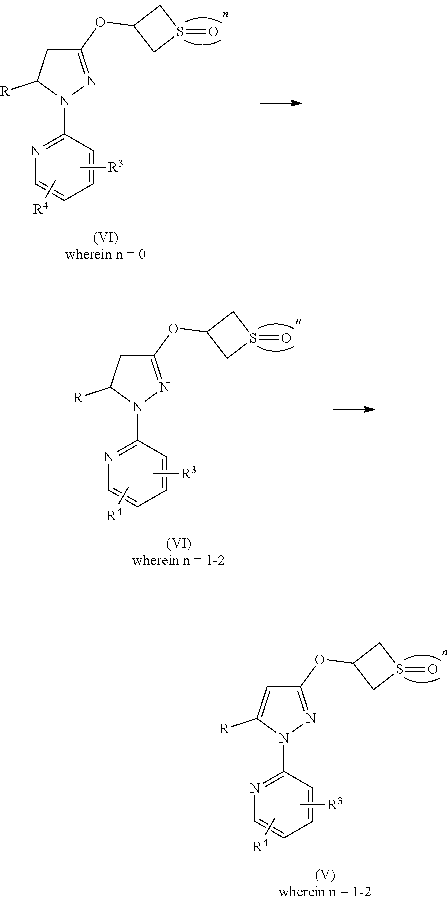

wherein, R, R³ and R⁴ are as defined herein above;
   or
1. converting a compound of formula (VI) to a compound of formula (V) wherein n=0 by reacting with a suitable acid, which upon further oxidation using a suitable oxidizing agent provides a compound of formula (V) wherein n=1 or 2;

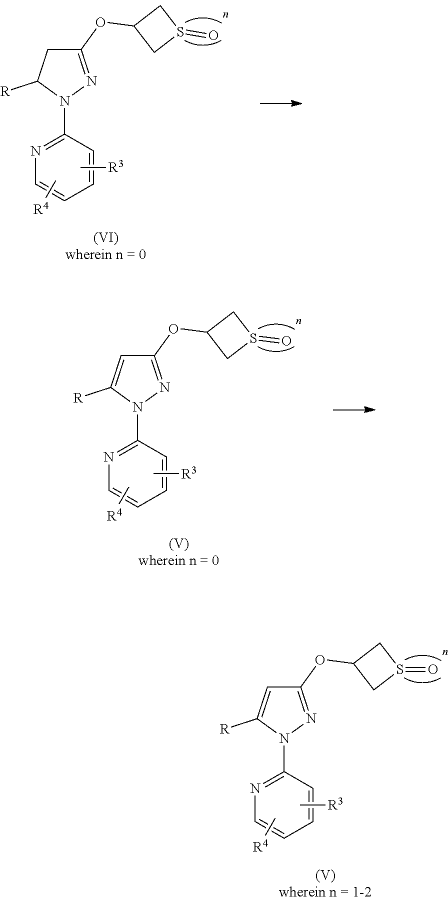

wherein, R, R³ and R⁴ are as defined herein above.

In one embodiment, the present invention provides a process for preparing a compound of formula (IV) wherein R′ represent COX, from a compound of formula (IV) wherein R′ represents COOH using a suitable halogenating agent.

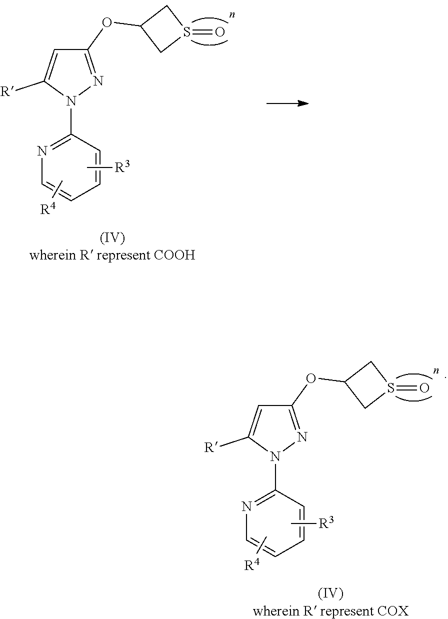

The suitable halogenating agent used for above conversion is selected from SOCl₂, SO₂Cl₂, COCl₂, X₂, C(═O)(OCl₃)₂, methanesulfonyl chloride, POX₃, PX₃, PX₅ or metal halides; wherein X is Cl or Br.

In a preferred embodiment, the compound of formula (VII) is reacted with a compound of formula (X) to obtain a compound of formula (VI) using a suitable base, optionally in the presence of a suitable catalyst and a suitable ligand;

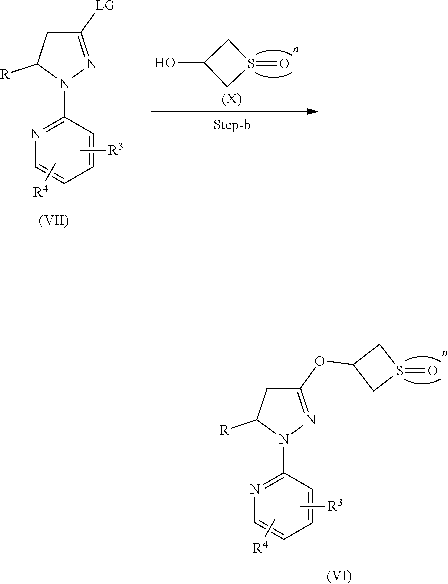

wherein, n=0-2, R, R³, R⁴ and LG are as defined herein above.

The suitable catalyst is selected in a non-limiting way from copper(I) iodide, copper(I) chloride, copper(II) chloride, iron(III) chloride (FeCl₃), copper(I) oxide, copper(II) acetate, copper(II) triflate, copper(I)-thiophene-2-carboxylate or DABCO®-CuCl compl.

The suitable ligand is selected in a non-limiting way from ethylene diamine (EDA), dimethyl ethylene diamine (DMEDA), tetramethylethylenediamine (TMEDA), dimethoxy ethane (DME), monoethylene glycol (MEG), acetyl acetone, ethylenediaminetetraacetic acid (EDTA), N,N-dimethyl formamide (DMF), thiophene-2-carboxylic acid, N,N-dimethyl glycine, L-proline, N-methyl-L-proline, 1,10-phenathroline (Phen), 2,2′-bipyridyl (bpy), 1,4-diazabicyclo[2.2.2]octane (DABCO), 2-acetylpyridine oxime or 1-methyl imidazole.

In one embodiment, the compound of formula (VII) is converted into a compound of formula (VI) using a suitable base optionally in the presence of a suitable phase transfer catalyst.

The phase transfer catalyst (PTC) is selected in a non-limiting way from tetrabutylammonium bromide (TBAB), tetrabutylammonium chloride (TBAC), tetrabutylammonium hydroxide (TBAH), Tetrabutylammonium fluoride (TBAF), tetrabutylammonium hydrogensulfate (TBA·HSO4), benzyltrimethylammonium hydroxide (Triton-B) or benzyltriethylammonium chloride (TEBA-Cl); preferably TBAB.

In one embodiment, the compound of formula (VII) is reacted with a compound of formula (X)

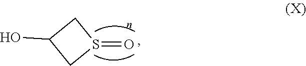

wherein n is 0, to obtain a compound of formula (VI).

In another embodiment, the compound of formula (VII) is reacted with a compound of formula (X)

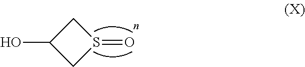

wherein n is 2, to obtain a compound of formula (VI).

In one embodiment, the compound of formula (VI) wherein n=0, is oxidized to the compound of formula (V) wherein n=0, using a suitable oxidizing agent;

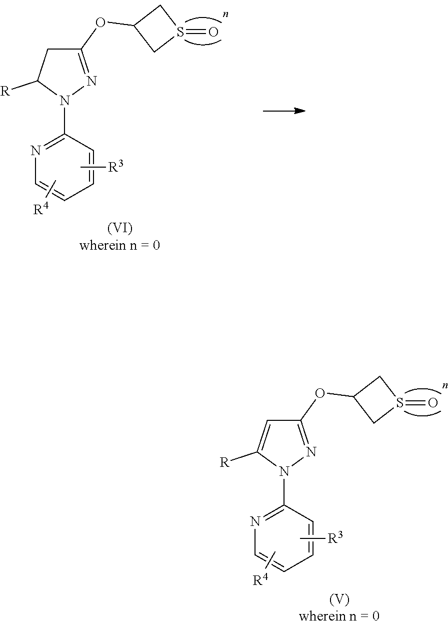

wherein, R is selected from CX₃, CN or COOR^{c}, R^{c} represents C₁-C₄ alkyl, and R³ and R⁴ are as defined herein above.

In another embodiment, the compound of formula (VI) wherein n=0, is oxidized to the compound of formula (V) wherein n=2, using a suitable oxidizing agent;

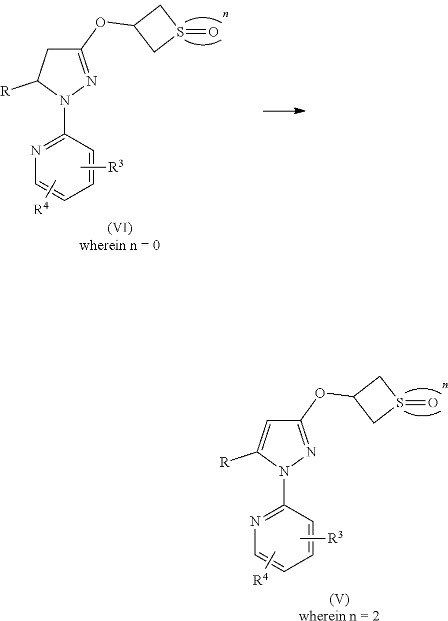

wherein, R is selected from CX₃, CN or COOR^{c}, R^{c} represents C₁-C₄ alkyl, and R³ and R⁴ are as defined herein above.

In yet another embodiment, the compound of formula (VI) wherein n=2, is oxidized to the compound of formula (V) wherein n=2 using a suitable oxidizing agent;

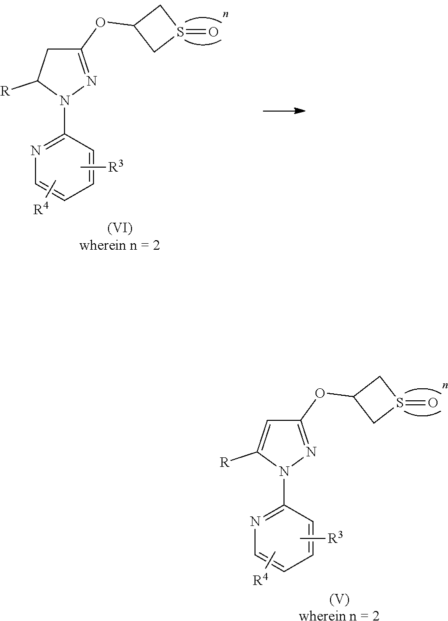

wherein, R is selected from CX₃, CN or COOR^{c}, R^{c} represents C₁-C₄ alkyl, R³ and R⁴ are as defined herein above.

In a preferred embodiment, the present invention provides a process for preparing compound of formula (IV) or salts thereof,
wherein,
n is 2;
R′ is COX or COOH;
R³ is halogen;
R⁴ is hydrogen.

In one embodiment, the present invention provides a novel compound of formula (Z) or salts thereof,

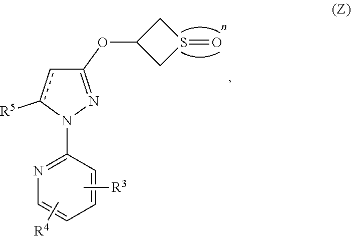

wherein,
R³ is selected from the group consisting of halogen, C₁-C₄ alkyl and C₁-C₄ haloalkyl;
R⁴ is selected from the group consisting of hydrogen, halogen, C₁-C₄ alkyl and C₁-C₄ haloalkyl;
R⁵ is selected from the group consisting of CX₃, CN, COOH, COX or COOR^{c};
   R^{c} represents C₁-C₄ alkyl;
   X represents halogen;
n represents an integer selected from 1 to 2;
[symbol] represents double or single bond.

In one embodiment, the present invention provides a process for preparing compounds of formula (I) or salts thereof,

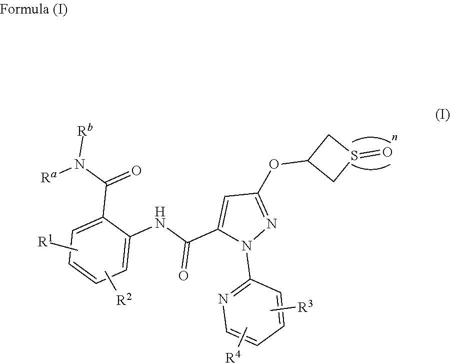

wherein,
R^{a} and R^{b} are independently selected from the group consisting of hydrogen, C₁-C₆ alkyl, C₃-C₆ cycloalkyl and C₃-C₆ cycloalkyl-C₁-C₄ alkyl; wherein R^{a} and R^{b} optionally substituted with one or more halogen;
R¹ and R² are independently selected from the group consisting of hydrogen, halogen, cyano, C₁-C₆ alkyl, C₁-C₄ haloalkyl, and C₃-C₆ cycloalkyl;
R³ is the group consisting of halogen, C₁-C₄ alkyl and C₁-C₄ haloalkyl;
R⁴ is the group consisting of hydrogen, halogen, C₁-C₄ alkyl and C₁-C₄ haloalkyl;
n represents an integer selected from 0 to 2;
   comprising the steps of:
a) obtaining a compound of formula (VII) from a compound of formula (VIII);

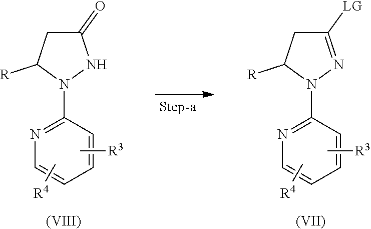

wherein, LG is selected from halogen, OMs, OTf, OTs; R is selected from CX₃, CN or COOR^{c}, R^{c} represents C₁-C₄ alkyl, R³ and R⁴ are as defined herein above;
b) reacting the compound of formula (VII) with a compound of formula (X) to obtain a compound of formula (VI);

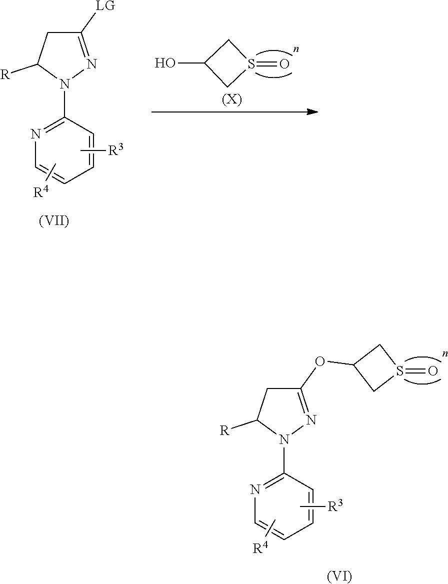

wherein n=0-2, R, R³, R⁴ and LG are as defined herein above;
c) converting the compound of formula (VI) to a compound of formula (V) using a suitable oxidizing agent and a suitable acid;

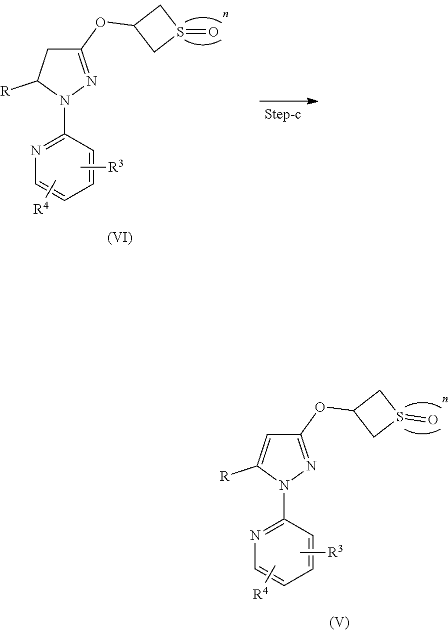

wherein n, R, R³ and R⁴ are as defined herein above;
d) converting the compound of formula (V) to a compound of formula (IV);

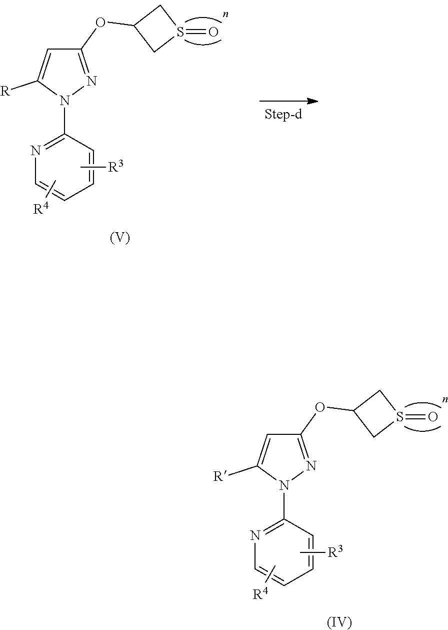

wherein R′ represents COOH or COX; n, R, and R³ and R⁴ are as defined herein above;
e) reacting the compound of formula (IV) with a compound of formula (III) and a suitable amine of formula (R^{a}R^{b}NH) or with a compound of formula (IIIa) to obtain a compound of formula (I);

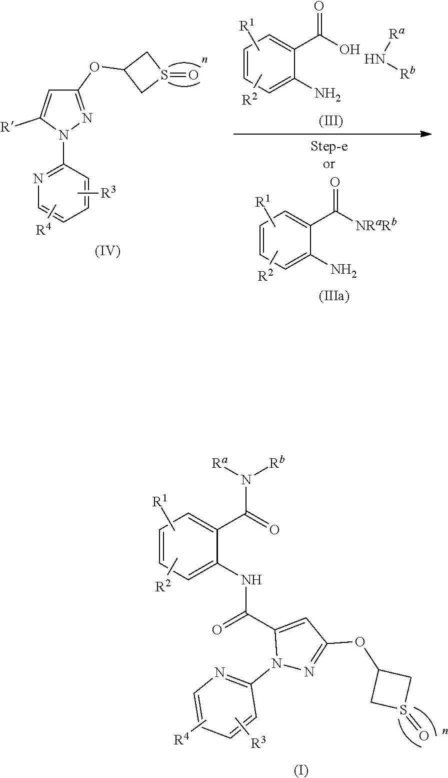

wherein n, R′, R^{a}, R^{b}, R¹, R², R³ and R⁴ are as defined herein above;

### Alternatively,

e) reacting the compound of formula (IV) with a compound of formula (III) and optionally isolating the compound of formula (IIa), followed by reacting with a suitable amine of the formula (R^{a}R^{b}NH), to obtain a compound of formula (I);

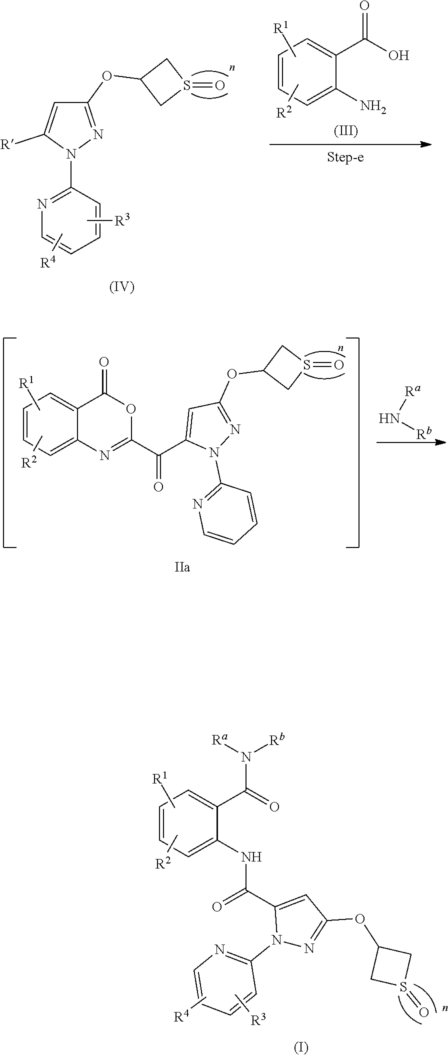

wherein n, R′, R^{a}, R^{b}, R¹, R², R³ and R⁴ are as defined herein above.

In another embodiment, the present invention provides a process for the preparation of a compound of formula (I) or a salt thereof,

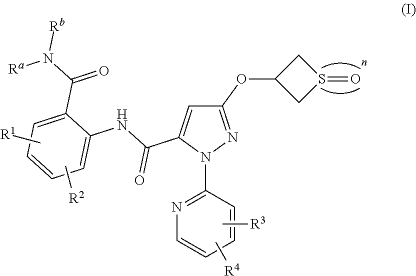

wherein,
n, R^{a}, R^{b}, R¹, R², R³ and R⁴ are as defined herein above;
   comprising the steps of:
a) oxidizing a compound of formula (VIII) using a suitable oxidizing agent or a suitable acid to obtain a compound of formula (IX);

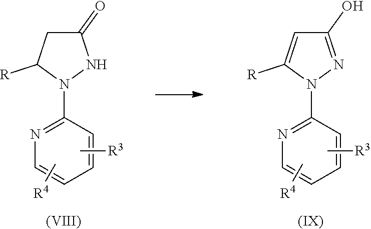

wherein, R is selected from CX₃, CN or COORS, R^{c} represents C₁-C₄ alkyl; R³ and R⁴ are as defined herein above;
b) converting the compound of formula (IX) with a compound of formula (X) to obtain a compound of formula (V);

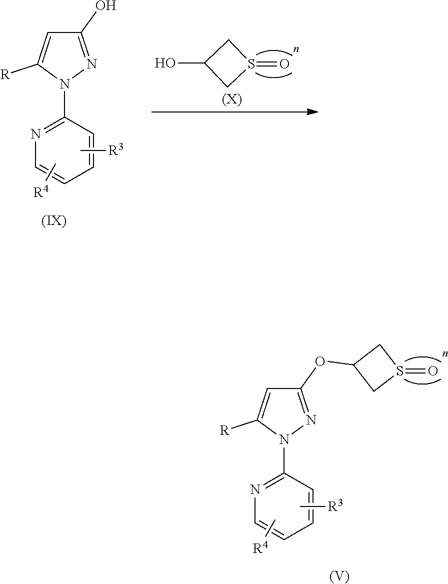

wherein, R, R³, R⁴ are as defined herein above;
c) converting the compound of formula (V) to a compound of formula (IV) in the presence of a suitable acid;

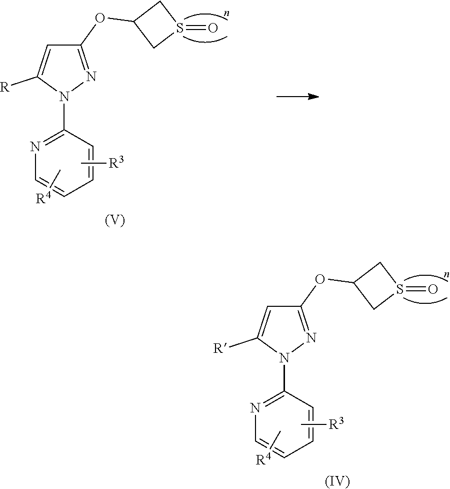

wherein, R′ represent COOH or COX, R, R³ and R⁴ are as defined herein above;
d) reacting the compound of formula (IV) with a compound of formula (III) and a suitable amine of formula (R^{a}R^{b}NH) or with a compound of formula (IIIa) to obtain a compound of formula (I);

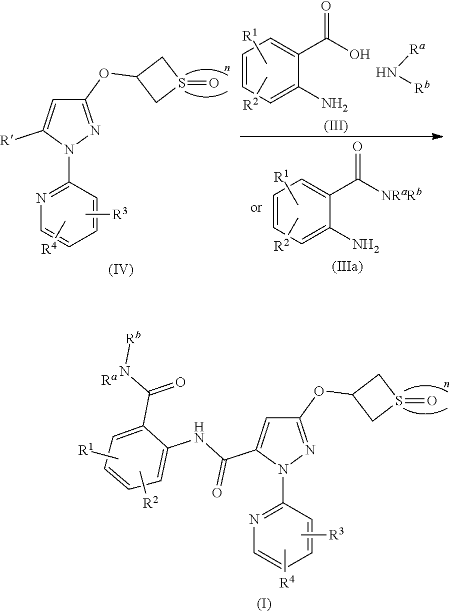

wherein, R′, R^{a}, R^{b}, R¹, R², R³ and R⁴ are as defined herein above.

In a preferred embodiment, the present invention provides a process for preparing a compound of formula (I) or a salt thereof,

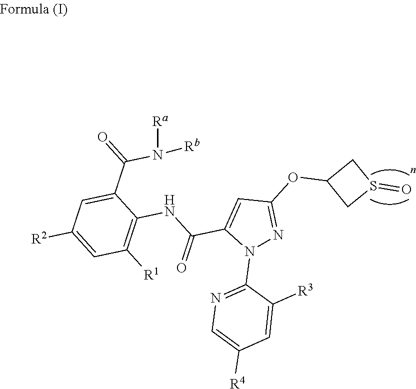

wherein,
R^{a} and R^{b} are independently selected from the group consisting of hydrogen, C₁-C₆ alkyl, C₃-C₆ cycloalkyl and C₃-C₆ cycloalkyl-C₁-C₄ alkyl; wherein R^{a} and R^{b} optionally substituted with one or more halogen;
R¹ and R² are independently selected from the group consisting of hydrogen, halogen, cyano, C₁-C₆ alkyl, C₁-C₄ haloalkyl, and C₃-C₆ cycloalkyl;
R³ is selected from the group consisting of halogen, C₁-C₄ alkyl and C₁-C₄ haloalkyl;
R⁴ is selected from the group consisting of hydrogen, halogen, C₁-C₄ alkyl and C₁-C₄ haloalkyl;
n represents an integer 2;
   from the compound of formula (IV) as prepared in claim 1,
   comprising the steps of:
a) obtaining a compound of formula (VIIa) from a compound of formula (VIIIa);

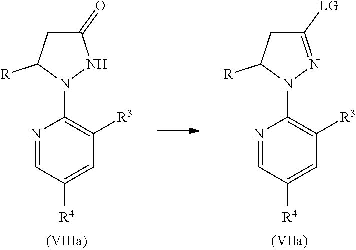

wherein, LG is selected from the group consisting of halogen, OMs, OTf and OTs; R is selected from the group consisting of CX₃, CN and COORS, Re represents C₁-C₄ alkyl; R³ and R⁴ are as defined herein above;
b) reacting the compound of formula (VIIa) with a compound of formula (X) to obtain a compound of formula (VIa), in the presence of suitable base and a suitable solvent;

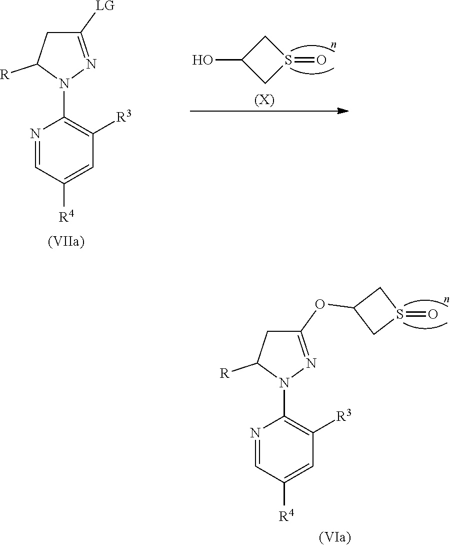

wherein, n=0 to 2; R³, R⁴, R and LG are as defined herein above;
c) converting the compound of formula (VIa) to a compound of formula (Va), in the presence of a suitable oxidizing agent and a suitable solvent;

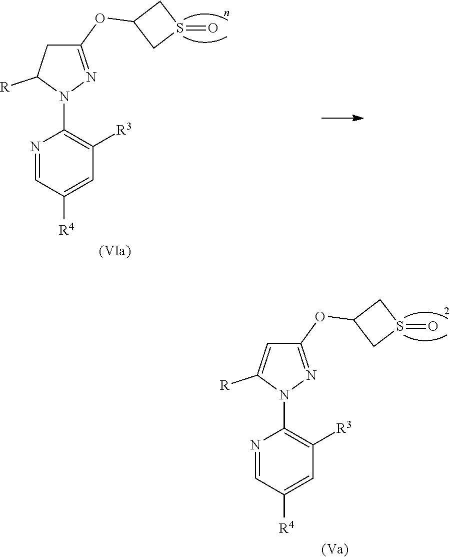

wherein, n=0 to 2; R, R³ and R⁴ are as defined herein above;
d) converting the compound of formula (Va) to a compound of formula (IVa);

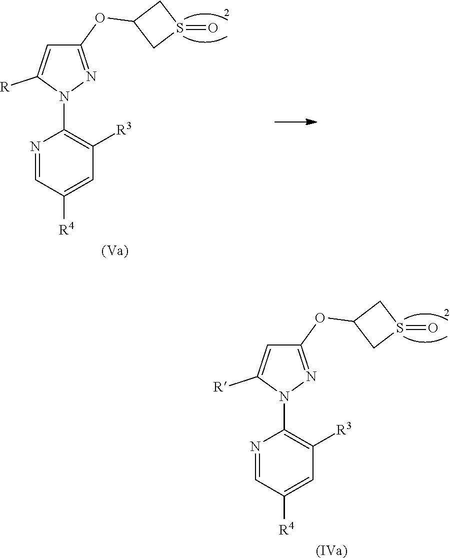

wherein R′ represents COOH or COX; R³, R⁴ and R are as defined herein above;
e) reacting the compound of formula (IVa) with a compound of formula (III-1) to obtain a compound of formula (IIa);

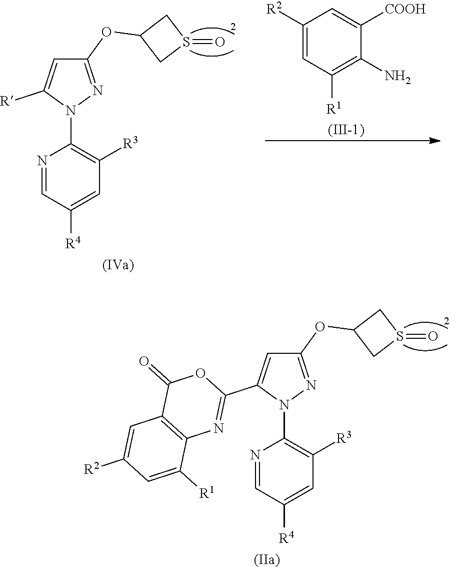

wherein, R′, R¹, R², R³ and R⁴ are as defined herein above;
or
reacting a compound of formula (IVa) with a compound of formula (IIIa-1) to obtain a compound of formula (I);

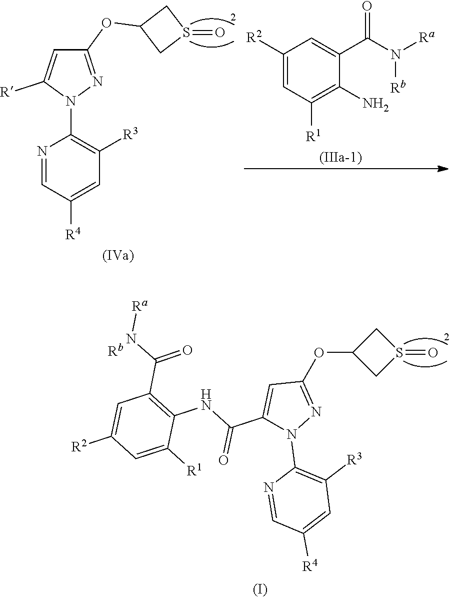

wherein, R′, R^{a}, R^{b}, R¹, R², R³ and R⁴ are as defined herein above.
f) reacting a compound of formula (IIa) with an amine (R^{a}R^{b}NH) to obtain a compound of formula (Ia)

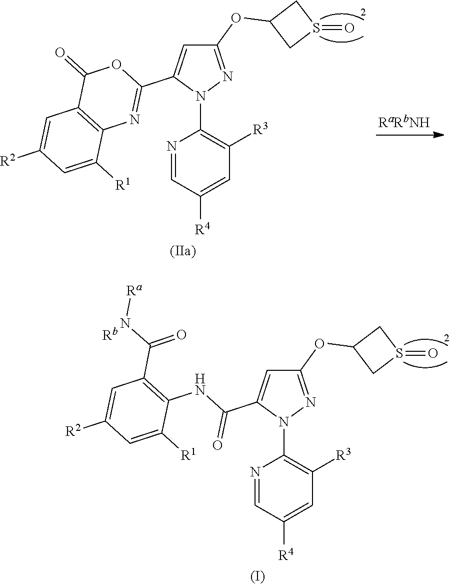

wherein, R^{a}, R^{b}, R¹, R², R³ and R⁴ are as defined herein above.

In a preferred embodiment, the present invention provides a process for preparation of compound of formula (I)
wherein,
n is 2;
R′ is selected from the group consisting of COX or COOH;
R^{a} and R^{b} are independently selected from the group consisting of hydrogen, C₁-C₆ alkyl, and C₃-C₆ cycloalkyl-C₁-C₄ alkyl;
R¹ and R² are independently selected from the group consisting of hydrogen, halogen, cyano, C₁-C₆ alkyl;
R³ is halogen;
R⁴ is hydrogen.
   In one of the particular embodiments, the present invention relates to a novel process for preparing a compound of formula (I); wherein, R^{a} and R^{b} are independently H, C₁-C₄ alkyl; R¹ is CH₃; R² is Cl; R³ is Cl and R⁴ is H.

The suitable leaving group (LG) mentioned in the reaction wherein a compound of formula (VII) is converted to a compound of formula (VI), is selected from halogen, OMs, OTf or OTs. Preferably, the suitable leaving group is halogen selected from Cl, Br or OMs; more preferably it is Cl or Br; most preferably it is Cl.

The suitable halogenating agent is selected from phosphoryl chloride, phosphoryl bromide, phosphorus trichloride, phosphorus pentachloride, methanesulfonyl chloride, tosyl chloride, bromine, chlorine, thionyl chloride, oxalyl chloride, CX₄—PPh₃, phosgene and cyanuric chloride.

In one embodiment, the halogenating agent is phosphoryl chloride.

In another embodiment, the halogenating agent is phosphoryl bromide.

The suitable base used in the process can be an organic or inorganic base.

The suitable inorganic base is selected from but is not limited to alkali metal hydrogen carbonates, such as lithium hydrogen carbonate (LiHCO₃), sodium hydrogen carbonate (NaHCO₃), potassium hydrogen carbonate (KHCO₃), and cesium hydrogen carbonate (CsHCO₃); alkali/alkaline earth metal carbonates such as sodium carbonate (Na₂CO₃), calcium carbonate (CaCO₃), cesium carbonate (Cs₂CO₃), lithium carbonate (Li₂CO₃), potassium carbonate (K₂CO₃); alkali/alkaline earth metal hydroxides such as lithium hydroxide (LiOH), sodium hydroxide (NaOH), potassium hydroxide (KOH), cesium hydroxide (CsOH), calcium hydroxide (Ca(OH)₂), alkali metal phosphates such as sodium diphosphate (Na₂HPO₄), sodium phosphate (Na₃PO₄), potassium diphosphate (K₂HPO₄), potassium phosphate (K₃PO₄); alkali metal halides such as sodium fluoride (NaF), potassium fluoride (KF), and cesium fluoride (CsF); alkali metal hydrides such as lithium hydride (LiH), sodium hydride (NaH), and potassium hydride (KH); and alkali metal alkoxides such as sodium methoxide (NaOCH₃), sodium ethoxide (NaOCH₂CH₃), sodium tert-butoxide and potassium tert-butoxide and the like.

The organic base is selected from amines which include but are not limited to ethylamine, triethylamine, isopropylamine diisopropylamine, triisopropylamine, pyridine, piperidine, methylmorpholine, N-methylpiperidine N,N-(dimethylamino)pyridine (DMAP), lutidine, collidine, tetramethylammonium hydroxide, tetrabutylammonium hydroxide, choline hydroxide; amidines which includes but is not limited to, 1,5,7-triazabicyclo[4.4.0]dec-5-ene, (TBD), 2,3,4,6,7,8,9,10-octahydropyrimidol[1,2-a]azepine (DBU) 1,5-diazabicyclo[4.3.0]non-5-ene (DBN), 1,4-diazabicyclo[2.2.2]octane (DABCO, triethylenediamine).

The suitable catalyst used in the process of the present invention is selected in a non-limiting way from copper chloride, copper iodide, trisodium or tripotassium phosphate, tetramethylethylenediamine, ethylenediamine and ferric chloride.

The suitable oxidizing agent is selected from manganese dioxide (MnO₂), potassium permanganate (KMnO₄), nitric acid (HNO₃), sodium nitrite (NaNO₃), activated charcoal, palladium on carbon, copper(I) chloride, copper(II) chloride, iron(III) chloride (FeCl₃), copper(II) acetate, oxygen, hydrogen peroxide, tertiary butyl hydrogen peroxide (TBHP), sulfuric acid, oxone, H₂O₂—AcOH, V₂P₅—H₂O₂, selenioum dioxide, selenous acid and CuCl—AcOH.

In a preferred embodiment, the oxidizing agent as used in the instant process of the present invention is selected in a not limiting way from nitric acid (HNO₃), H₂O₂—AcOH, V₂O₅—H₂O₂ and potassium permanganate.

According to an embodiment, the compound of formula (V) is converted to a compound of formula (IV) by hydrolysis using a suitable hydrolyzing agent.

The suitable hydrolyzing agent used in the process is an acid and said acid is selected in a non-limiting way from aqueous sulfuric acid (aq H₂SO₄) and perchloric acid and hydrochloric acid (HCl).

In a preferred embodiment, the hydrolyzing agent used is 10-50% aqueous sulfuric acid, more preferably 20% aqueous H₂SO₄.

Optionally, the hydrolysis of the compound of formula (V) to obtain a compound of formula (IV) can also be carried out in the presence of acid supported ion exchange resins or an acidic zeolites.

The suitable solvents used in steps (a) to (e) are selected in a non-limiting way from the group consisting of aliphatic, alicyclic or aromatic hydrocarbons, halogenated hydrocarbons, ethers, nitriles, amides, alcohols or of combinations thereof.

The suitable solvents used in steps (a) to (e) are selected in a non-limiting way from the group consisting of acetonitrile, acetic acid, acetone, hexane, heptane, octane, nonane, decane, dodecane, cycloalkanes: cyclopropane, cyclobutane, cyclopentane, cyclohexane, cycloheptane, cyclooctane; dimethyl formamide, ethylene dichloride, ethyl acetate, toluene, xylene, mesitylene, benzene, diisopropyl ether, t-butyl methyl ether, tetrahydrofuran, 2-methyl tetrahydrofuran, dioxane, monoglyme, diglyme, methoxy-methane, methoxy-ethane ethoxy-ethane, di-methoxyethane, di-ethoxyethane, dichloromethane, chloroform, dichloroethane, N,N-dimethylmethanamide, dimethyl sulfoxide, N-methyl-2-pyrrolidone, 1,3-dimethyl-3,4,5,6-tetrahydro-2(1H)-pyrimidinone, hexamethylphosphoramidem, 1,3-dimethyl-2-imidazolidinone or of combinations thereof.

In an embodiment, steps (a) to (e) are carried out using a solvent selected from acetonitrile, acetone, N,N-dimethyl formamide, ethylene dichloride, ethyl acetate, toluene and pyridine.

The reaction time is not critical and depends on the batch size, the temperature, the reagents and solvents being employed. Typically, the reaction time may vary from a few minutes to several hours.

The process step (a) is preferably carried out at a temperature in the range of 70° C. to 110° C. It is also possible to carry out this reaction at higher or lower temperatures.

The process step (b) is preferably carried out at a temperature in the range of 80° C. to 130° ° C. It is also possible to carry out this reaction at higher or lower temperatures.

The process step (c) is carried out at a temperature in the range of 0° C. to 70° ° C. It is also possible to carry out this reaction at higher or lower temperatures.

The process step (d) is carried out at a temperature in the range of 70° C. to 120° ° C. It is also possible to carry out this reaction at higher or lower temperatures.

The process step (e) is carried out at a temperature in the range of 0° ° C. to 70° ° C. It is also possible to carry out this reaction at higher or lower temperatures.

In a preferred embodiment, step-a (halogenation) is carried out in the presence of a suitable halogenating reagent selected in a non-limiting way from phosphorus oxychloride (POCl₃), phosphorus oxybromide (POBr₃), methane sulfonyl chloride (MsCl), para-toluyl sulphonyl chloride (p-TSCl), and triflic anhydride (Tf₂O), and a suitable base selected in a non-limiting way from triethylamine (Et₃N), diisopropyl ethylamine (DIPEA), potassium carbonate (K₂CO₃), sodium carbonate (Na₂CO₃), tripotassium phosphate (K₃PO₄), and trisodium phosphate (Na₃PO₄)

In another preferred embodiment, step-b (etherification) is carried out in the presence of a suitable base selected in a non-limiting way from triethylamine (Et₃N), diisopropyl ethylamine (DIPEA), N,N-dimethyl guanidine (DMG), pyridine, 3-methyl pyridine, sodium hydride (NaH), sodium hydroxide (NaOH), potassium hydroxide (KOH), sodium tertiary butoxide (tBuONa), potassium tertiary butoxide (tBuOK), cesium carbonate (Cs₂CO₃), potassium carbonate (K₂CO₃), sodium carbonate (Na₂CO₃), sodium bicarbonate (NaHCO₃), tripotassium phosphate (K₃PO₄), dipotasium hydrogen phosphate (K₂HPO₄), monopotasium hydrogen phosphate (KH₂PO₄), trisodium phosphate (Na₃PO₄), disodium phosphate (Na₂HPO₄), monosodium phosphate (NaH₂PO₄), calcium carbonate (CaCO₃), sodium ethoxide, and sodium bis(trimethylsilyl)amide (NaHMDS); more preferably sodium hydroxide or tripotassium phosphate (K₃PO₄), and a suitable catalyst selected in a non-limiting way from copper(—I) iodide, copper(I) chloride, copper(II) chloride, iron(III) chloride (FeCl₃), copper(I) oxide, copper(II) acetate, copper(II)triflate, copper(I)-thiophene-2-carboxylate, DABCO®-CuCl complex; a suitable ligand selected in a non-limiting way from ethylene diamine (EDA), dimethyl ethylene diamine (DMEDA), tetramethylethylenediamine (TMEDA), dimethoxy ethane (DME), monoethylene glycol (MEG), acetyl acetone, ethylenediaminetetraacetic acid (EDTA), N,N-dimethyl formamide (DMF), thiophene-2-carboxylic acid, N,N-dimethyl glycine, L-proline, N-methyl-L-proline, 1,10-phenathroline (Phen), 2,2′-bipyridyl (bpy), 1,4-diazabicyclo[2.2.2]octane (DABCO), 2-acetylpyridine oxime, and 1-methyl imidazole; and a suitable solvent selected in a non-limiting way from toluene, xylenes, chlorobenzene, o-dichlorobenzene (ODCB), N,N′-dimethyl formamide (DMF), dimethoxy ethane (DME), ethylacetate (EtOAc), n-butyl acetate, Ethanol, Acetonitrile (MeCN), sulfolane, dimethyl sulfoxide (DMSO), tetrahydrofuran (THF), 2-methyl tetrahydrofuran (2-Me THF), cyclohexane, dimethyl carbonate (DMC), and 1,2-dichloroethane (DCE)

In yet another preferred embodiment, step-c (oxidation) is carried out in the presence of a suitable oxidizing agent selected in a non-limiting way from manganese dioxide (MnO₂), potassium permanganate (KMnO₄), nitric acid (HNO₃), sodium nitrite (NaNO₃), activated charcoal, palladium on carbon, copper(I) chloride, copper(II) chloride, iron(III) chloride (FeCl₃), copper(II) acetate, oxygen, hydrogen peroxide, tertiary butyl hydrogen peroxide (TBHP), sulfuric acid; a suitable catalyst selected in a non-limiting way from sodium tungstate, tungstic acid, trifluroacetic acid, acetic acid, selenium dioxide, selenous acid, vanadium pentoxide (V₂O₅); more preferably sodium tungstate, tungstic acid; a suitable solvent selected in a non-limiting way from ethyl acetate, toluene, xylenes, chlorobenzene, N,N-dimethyl formamide (DMF), dimethyl sulfoxide (DMSO), acetonitrile (MeCN), sulfolane, tetrahydrofuran (THF), and 1,2-dichloroethane (DCE)

In yet another preferred embodiment, step-d (hydrolysis) is carried out in the presence of a suitable acid selected in a non-limiting way from hydrochloric acid, sulfuric acid, Amberlyst®-15, polyphosphoric acid, phosphoric acid, camphor sulfonic acid, and formic acid, and a suitable solvent selected from acetic acid, water, THF, and ethanol.

In yet another preferred embodiment, step-e (cyclization) is carried out in the presence of a suitable base selected in a non-limiting way from triethyl amine, diisopropyl ethylamine, pyridine, 3-methyl pyridine, 2,6-lutidine; and a suitable solvent selected from acetonitrile (MeCN), 1,2-dichloroethane (DCE), dichloromethane (DCM). In yet another preferred embodiment, step-f (amidation) is carried out in the presence of a suitable solvent selected in a non-limiting way from N,N-dimethyl formamide (DMF), dimethyl sulfoxide (DMSO), acetonitrile (MeCN), isopropanol (IPA), acetone, N,N-dimethyl acetamide (DMAc), and acetic acid (AcOH).

Any person skilled in the art knows the best work-up of the reaction mixtures after the end of the respective reactions. In one embodiment, the work-up is usually carried out by isolation of the product, and optionally washing with solvent, and further optionally drying of the product if useful or required.

The isolation of the reaction product can be carried out by a technique which includes but is not limited to decantation, filtration, centrifugation, evaporation, liquid-liquid extraction, distillation, recrystallization, chromatography and the like or a combination thereof.

The process steps according to the invention are generally carried out under atmospheric pressure. Alternatively, however, it is also possible to work under reduced pressure or higher pressure.

In the context of the present invention, the term “optionally” when used in reference to any element, to intermediates, reagents or conditions, including any process step, e.g., the isolation of intermediates; is intended to mean that the subject element is isolated, or alternatively is not isolated from the reaction mixture and directly used for the subsequent chemical reaction.

Similarly, this definition is applied in case for reagents or reaction conditions as well.

The specification herein and the various features and advantageous details thereof are explained with reference to the non-limiting examples in the description. Descriptions of well-known components and processing techniques are omitted so as to not unnecessarily obscure the embodiments herein. The examples used herein are intended merely to facilitate an understanding of ways in which the specification herein may be practiced and to further enable those of skilled in the art to practice the specification herein. Accordingly, the examples should not be construed as limiting the scope of the specification herein.

Any discussion of documents, acts, materials, devices, articles and the like that has been included in this specification is solely for the purpose of providing a context for the disclosure. It is not to be taken as an admission that any or all of these matters form a part of the prior art base or were common general knowledge in the field relevant to the disclosure as it existed anywhere before the priority date of this application.

The numerical values mentioned in the description and the foregoing claims though might forming a critical part of the present invention of the present disclosure, as well as any deviation from such numerical values shall still fall within the scope of the present disclosure if that deviation follows the same scientific principle as that of the present invention disclosed in the present disclosure.

The invention is further illustrated by the following examples which are provided to be exemplary of the invention, and do not limit the scope of the invention. While the present invention has been described in terms of its specific embodiments, certain modifications and equivalents will be apparent to those skilled in the art and are intended to be included within the scope of the present invention.

### CHEMISTRY EXAMPLES

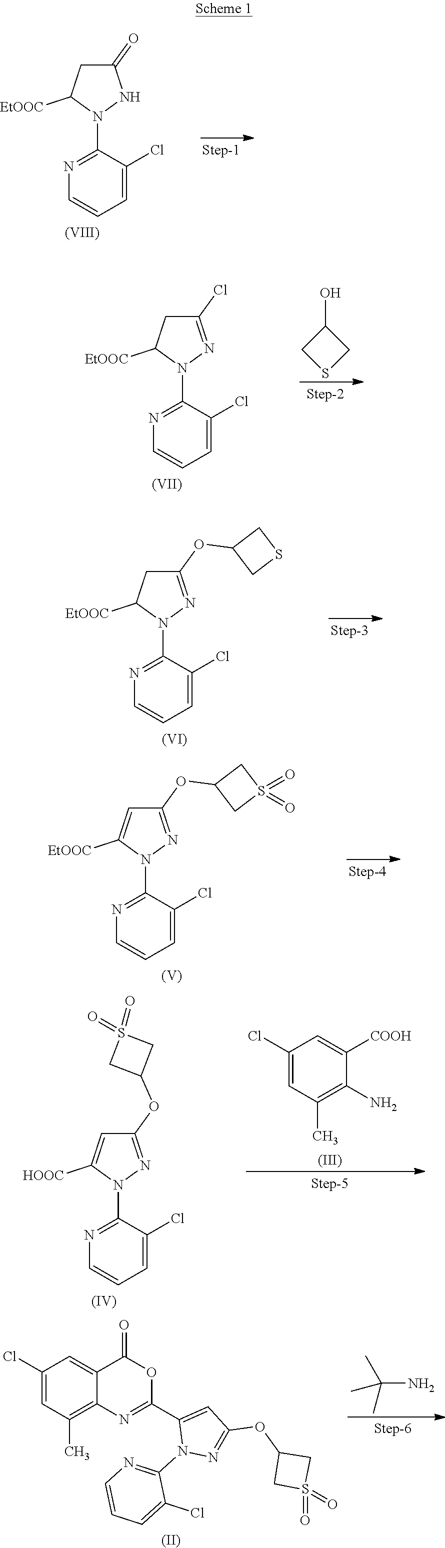

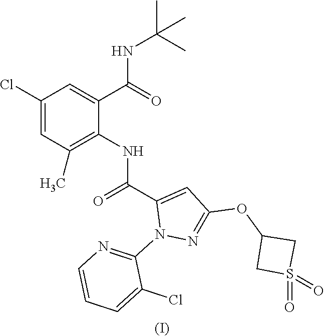

### Example 1: Preparation of Ethyl 3-chloro-1-(3-chloropyridin-2-yl)-4,5-dihydro-1H-pyrazole-5-carboxylate

### Step 1 Process-1: Ethyl 3-chloro-1-(3-chloropyridin-2-yl)-4,5-dihydro-1H-pyrazole-5-carboxylate

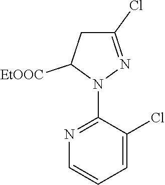 [SMILES: CCOC(=O)C1CC(Cl)=NN1c1ncccc1Cl]

To a stirred solution of ethyl 2-(3-chloropyridin-2-yl)-5-oxopyrazolidine-3-carboxylate (250 g, 0.93 mol) in 1, 2-dichloroethane (DCE) (2000 mL), N, N-dimethylformamide (0.68 g, 0.01 mol) was added, followed by drop wise addition of phosphorous oxychloride (170.6 g, 1.11 mol). The reaction mixture was heated to 80-85° ° C. and maintained at the same temperature for 7 h. After completion of the reaction, the reaction mixture was cooled to 25-30° C. and quenched by slowly pouring it into water (1250 mL). The aqueous layer was extracted with DCE (2×500 mL). The combined DCE layers were washed with a brine solution (500 mL), dried over anhydrous sodium sulphate, and concentrated under reduced pressure to obtain a crude oily product. Isopropanol (125 mL) was added to the crude product and co-distilled completely off under reduced pressure. The residue obtained was dissolved in isopropanol (375 mL) and the solution was cooled to 25-30° ° C. to obtain a solid. Water (1875 mL) was added, and the resulting mixture was stirred further for 3-4 h. The solid product was filtered, the wet filter cake was washed with water (500 mL) and dried under reduced pressure to obtain ethyl 3-chloro-1-(3-chloropyridin-2-yl)-4,5-dihydro-1H-pyrazole-5-carboxylate (240 g, 90% yield).

¹H NMR (DMSO-d₆, 400 MHz): 8.11-8.10 (dd, J=1.6 Hz, 1H), 7.84-7.81 (dd, J=1.6 Hz, 1H), 6.99-6.96 (dd, J=4.8 Hz, 1H), 5.24 (m, 1H), 4.10 (q, 2H), 3.55 (m, 1H), 3.26 (m, 1H), 1.11 (t, 3H); MS: m/ [symbol] =288.25 [M+H]

### Process-2: Ethyl 3-chloro-1-(3-chloropyridin-2-yl)-4,5-dihydro-1H-pyrazole-5-carboxylate

To a stirred solution of ethyl 2-(3-chloropyridin-2-yl)-5-oxopyrazolidine-3-carboxylate (800 g, 2.966 mol) in 1, 2-dichloroethane (DCE) (6400 mL), N, N-dimethylformamide (2.2 g, 29.7 mmol) was added, followed by drop wise addition of phosphorous oxychloride (546 g, 3.56 mol). The reaction mixture was heated to 80-85° C. and maintained at the same temperature for 7 h. After completion of the reaction, the reaction mixture was cooled to 25-30° C. and quenched by slowly pouring it into water (4000 mL). The aqueous layer was washed with DCE (3200 mL). The DCE layer was washed with brine solution (1600 mL), dried over anhydrous sodium sulphate and concentrated under reduced pressure to obtain a crude oily product. Isopropanol (400 mL) was added to the crude product and co-distilled completely under reduced pressure. The residue obtained was dissolved in isopropanol (1200 mL) and the solution was cooled to 25-30° C. to obtain a solid. Water (6000 mL) was added, and the resulting mixture was stirred further for 3-4 h. The solid product was filtered, the wet cake was washed with water (1600 mL) and dried under reduced pressure to obtain pure ethyl 3-chloro-1-(3-chloropyridin-2-yl)-4,5-dihydro-1H-pyrazole-5-carboxylate (800 g, 94% yield, HPLC purity 99%).

### Process-3: Ethyl 3-chloro-1-(3-chloropyridin-2-yl)-4,5-dihydro-1H-pyrazole-5-carboxylate

To a stirred solution of ethyl 2-(3-chloropyridin-2-yl)-5-oxopyrazolidine-3-carboxylate (5 g, 18.54 mol) in toluene (40 mL), phosphorous oxychloride (3.4 g, 22.25 mmol) was added drop wise. The reaction mixture was heated to 105-110° C. and maintained at the same temperature for 3 h. After completion of the reaction, the reaction mixture was cooled to 25-30° C. and quenched by slowly pouring into water (30 mL). The reaction mass was neutralized by 10% sodium bicarbonate solution (150 mL). The organic layer was separated, and the aqueous layer was extracted with toluene (2×25 mL). The combined toluene layers were washed with brine solution (50 mL), dried over anhydrous sodium sulphate and concentrated under reduced pressure to obtain crude ethyl 3-chloro-1-(3-chloropyridin-2-yl)-4,5-dihydro-1H-pyrazole-5-carboxylate (4.2 g, 79% crude yield).

### Process-4: Ethyl 3-chloro-1-(3-chloropyridin-2-yl)-4,5-dihydro-1H-pyrazole-5-carboxylate

To a stirred solution of ethyl 2-(3-chloropyridin-2-yl)-5-oxopyrazolidine-3-carboxylate) (5 g, 18.54 mol) in toluene (40 mL), phosphorous oxychloride (3.4 g, 22.25 mmol) was added drop wise followed by N, N-dimethylformamide (cat.). The reaction mixture was heated to 80-85° C. and maintained at the same temperature for 10 h. After completion of the reaction, the reaction mixture was cooled to 25-30° C. and quenched by slowly pouring into water (30 mL). The reaction mass was neutralized by 10% sodium bicarbonate solution (150 mL). The organic layer was separated, and the aqueous layer was extracted with DCE (2×25 mL). The combined organic layers were washed with brine solution (50 mL), dried over anhydrous sodium sulphate and concentrated under reduced pressure to obtain crude ethyl 3-chloro-1-(3-chloropyridin-2-yl)-4,5-dihydro-1H-pyrazole-5-carboxylate (4.8 g, 97% crude yield).

### Example 1a: Preparation of Ethyl 3-bromo-1-(3-chloropyridin-2-yl)-4,5-dihydro-1H-pyrazole-5-carboxylate

### Step 1: Ethyl 3-bromo-1-(3-chloropyridin-2-yl)-4,5-dihydro-1H-pyrazole-5-carboxylate

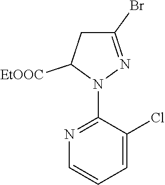 [SMILES: CCOC(=O)C1CC(Br)=NN1c1ncccc1Cl]

To a stirred solution of ethyl 2-(3-chloropyridin-2-yl)-5-oxopyrazolidine-3-carboxylate (1000 g, 3.708 mol) in 1,2-dichloroethane (DCE) (8000 mL), phosphorous oxychloride (1276 g, 4.450 mol) was added drop wise. The reaction mixture was heated to 80-85° C. and maintained at the same temperature for 7 h. After completion of the reaction, the reaction mixture was cooled to 25-30° C. and quenched by slowly pouring it into water (4000 mL). The reaction mass was neutralized with solid sodium bicarbonate (467 g, 5562 mmol). The aqueous layer was separated and washed with DCE (2×500 mL). The combined DCE layers were washed with brine solution (500 mL), dried over anhydrous sodium sulphate, and concentrated under reduced pressure to obtain a crude oily product. Isopropanol (2000 mL) was added to the crude product and stirred further for 16 h. The solid obtained was filtered, washed with IPA (500 mL) and dried under reduced pressure to obtain pure ethyl 3-bromo-1-(3-chloropyridin-2-yl)-4,5-dihydro-1H-pyrazole-5-carboxylate (953 g, 77% yield, HPLC purity 98%)

### Example 2: Preparation of Ethyl 1-(3-chloropyridin-2-yl)-3-(thietan-3-yloxy)-4,5-dihydro-1H-pyrazole-5-carboxylate

### Step 2: Process-1: Ethyl 1-(3-chloropyridin-2-yl)-3-(thietan-3-yloxy)-4,5-dihydro-1H-pyrazole-5-carboxylate Using CuI as a Catalyst and Cs₂CO₃ as a Base

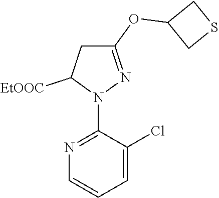 [SMILES: CCOC(=O)C1CC(OC2CSC2)=NN1c1ncccc1Cl]

To a stirred solution of ethyl 3-chloro-1-(3-chloropyridin-2-yl)-4,5-dihydro-1H-pyrazole-5-carboxylate (100 g, 0.30 mol) in toluene (1200 mL), copper (I) iodide (5.7 g, 0.03 mol), 1,10-phenanthroline (6.5 g, 0.04 mol), 3-thietanol (40.6 g, 0.45 mol) and cesium carbonate (147.0 g, 0.45 mol) were added at 40-45° C. under nitrogen atmosphere. The reaction mixture was stirred at 105-110° C. for 1-2 h. After completion of the reaction, the reaction mixture was cooled to 40-50° C. and filtered through a celite bed. The toluene layer was washed with brine solution (300 mL), dried over anhydrous sodium sulphate, and concentrated under reduced pressure at 45-50° C. to obtain a crude product. Isopropanol (100 mL) was added to the crude product and co-distilled off completely under reduced pressure. The residue obtained was dissolved in isopropanol (250 mL) at 40-45° C. and slowly cooled to 5-10° C. to obtain a solid product. The solid product was filtered, washed with isopropanol (50 mL) and dried under reduced pressure to obtain pure ethyl 1-(3-chloropyridin-2-yl)-3-(thietan-3-yloxy)-4,5-dihydro-1H-pyrazole-5-carboxylate (101 g, 85% yield)

¹H NMR (DMSO-d₆, 400 MHz): 8.04-8.02 (dd, J=1.6 Hz, 1H), 7.75-7.72 (dd, J=1.6 Hz, 1H), 6.85-6.82 (dd, J=4.8 Hz, 1H), 5.45 (m, 1H), 5.02 (m, 1H), 4.10 (q, 2H), 3.51 (m, 2H), 3.43 (m, 2H), 3.31 (m, 1H), 2.93 (m, 1H), 1.15 (t, 3H); MS: m/ [symbol] =342.15 [M+H]

### Process-2: Ethyl 1-(3-chloropyridin-2-yl)-3-(thietan-3-yloxy)-4,5-dihydro-1H-pyrazole-5-carboxylate Using K₃PO₄ as a Base

To a stirred solution of ethyl 3-chloro-1-(3-chloropyridin-2-yl)-4,5-dihydro-1H-pyrazole-5-carboxylate (20 g, 69.4 mmol) in toluene (240 mL), copper (I) iodide (1.32 g, 6.94 mmol), 1,10-phenanthroline (1.5 g, 8.33 mol), 3-thietanol (9.4 g, 104 mmol) and potassium triphosphate (44.2 g, 208 mmol) were added at 40-45° C. under nitrogen atmosphere. The reaction mixture was stirred at 105-110° C. for 2-4 h. After completion of the reaction, the reaction mixture was cooled to 40-50° C. and filtered through a celite bed. The toluene layer was washed with brine solution (60 mL), dried over anhydrous sodium sulphate, and concentrated under reduced pressure at 45-50° C. to obtain a crude product. Isopropanol (20 mL) was added to the crude product and co-distilled off completely under reduced pressure. The residue obtained was dissolved in isopropanol (50 mL) at 40-45° C. and slowly cooled to 5-10° C. to obtain a solid product. The solid product filtered, washed with isopropanol (10 mL) and dried under reduced pressure to obtain pure ethyl 1-(3-chloropyridin-2-yl)-3-(thietan-3-yloxy)-4,5-dihydro-1H-pyrazole-5-carboxylate (20.2 g, 85% yield).

¹H NMR (DMSO-d₆, 400 MHz): 8.04-8.02 (dd, J=1.6 Hz, 1H), 7.75-7.72 (dd, J=1.6 Hz, 1H), 6.85-6.82 (dd, J=4.8 Hz, 1H), 5.45 (m, 1H), 5.02 (m, 1H), 4.10 (q, 2H), 3.51 (m, 2H), 3.43 (m, 2H), 3.31 (m, 1H), 2.93 (m, 1H), 1.15 (t, 3H); MS: m/ [symbol] =342.15 [M+H]

### Process-3: Ethyl 1-(3-chloropyridin-2-yl)-3-(thietan-3-yloxy)-4,5-dihydro-1H-pyrazole-5-carboxylate using anhydrous FeCl; as a catalyst and K₃PO₄ as a base

To a stirred solution of ethyl 3-chloro-1-(3-chloropyridin-2-yl)-4,5-dihydro-1H-pyrazole-5-carboxylate (20 g, 69.4 mmol) in toluene (240 mL), anhydrous iron (III) chloride (2.25 g, 13.88 mmol), 1,10-phenanthroline (1.5 g, 8.33 mol), 3-thietanol (9.4 g, 104 mmol) and potassium triphosphate (58.95 g, 277.6 mmol) were added at 25-30° ° C. under a nitrogen atmosphere. The reaction mixture was stirred at 105-110° C. for 4-6 h. After completion of the reaction, the reaction mixture was cooled to 45-50° C. and filtered through a celite bed. The toluene layer was washed with brine solution (60 mL), dried over anhydrous sodium sulphate, and concentrated under reduced pressure to obtain crude ethyl 1-(3-chloropyridin-2-yl)-3-(thietan-3-yloxy)-4,5-dihydro-1H-pyrazole-5-carboxylate (20.8 g, 87% yield).

¹H NMR (DMSO-d₆, 400 MHz): 8.04-8.02 (dd, J=1.6 Hz, 1H), 7.75-7.72 (dd, J=1.6 Hz, 1H), 6.85-6.82 (dd, J=4.8 Hz, 1H), 5.45 (m, 1H), 5.02 (m, 1H), 4.10 (q, 2H), 3.51 (m, 2H), 3.43 (m, 2H), 3.31 (m, 1H), 2.93 (m, 1H), 1.15 (t, 3H); MS: m/ [symbol] =342.15 [M+H]

### Process-4: Ethyl 1-(3-chloropyridin-2-yl)-3-(thietan-3-yloxy)-4,5-dihydro-1H-pyrazole-5-carboxylate using anhydrous CuI as a catalyst and Cs₂CO₃ as a base

To a stirred solution of ethyl 3-chloro-1-(3-chloropyridin-2-yl)-4,5-dihydro-1H-pyrazole-5-carboxylate (150 g, 521 mmol) in toluene (1800 mL), copper(I) iodide (9.91 g, 52.1 mmol), 1,10-phenanthroline (11.26 g, 62.5 mmol), thietan-3-ol (70.4 g, 781 mmol) and cesium carbonate (254 g, 781 mmol) were added at 40-45° C. under nitrogen atmosphere. The reaction mixture was stirred at 105-110° C. for 1-6 h. After completion of the reaction, the reaction mixture was cooled to 40-50° C. and filtered through a celite bed. The toluene layer was washed with brine solution (300 mL), dried over anhydrous sodium sulphate, and concentrated under reduced pressure to obtain a crude product. Isopropanol (150 mL) was added to the crude product and co-distilled off completely under reduced pressure. The residue obtained was dissolved in isopropanol (150 mL) at 40-45° C. and slowly cooled to 20-30° C., followed by addition of DM water (450 mL). The solid obtained was filtered, washed with isopropanol (150 mL) and dried under reduced pressure to obtain pure ethyl 1-(3-chloropyridin-2-yl)-3-(thietan-3-yloxy)-4,5-dihydro-1H-pyrazole-5-carboxylate (154 g, 87% yield)

### Process-5: Ethyl 1-(3-chloropyridin-2-yl)-3-(thietan-3-yloxy)-4,5-dihydro-1H-pyrazole-5-carboxylate Using Anhydrous CuI as a Catalyst and K₃PO₄ as a Base

To a stirred solution of ethyl 3-chloro-1-(3-chloropyridin-2-yl)-4,5-dihydro-1H-pyrazole-5-carboxylate (20 g. 69.4 mmol) in toluene (240 mL), copper (I) iodide (1.32 g, 6.94 mmol), 1, 10-phenanthroline (1.5 g, 8.33 mol), 3-thietanol (8.13 g, 90 mmol) and potassium triphosphate (58.9 g, 278 mmol) were added at 40-45° C. under nitrogen atmosphere. The reaction mixture was stirred at 105-110° C. for 2-4 h. After completion of the reaction, the reaction mixture was cooled to 40-50° C. and filtered through a celite bed. The toluene layer was washed with brine solution (60 mL), dried over anhydrous sodium sulphate and concentrated under reduced pressure to obtain crude ethyl 1-(3-chloropyridin-2-yl)-3-(thietan-3-yloxy)-4,5-dihydro-1H-pyrazole-5-carboxylate (20 g, 84% yield).

### Process-6: Ethyl 1-(3-chloropyridin-2-yl)-3-(thietan-3-yloxy)-4,5-dihydro-1H-pyrazole-5-carboxylate Using Anhydrous CuI as a Catalyst and K₃PO₄ as a Base

To a stirred solution of ethyl 3-chloro-1-(3-chloropyridin-2-yl)-4,5-dihydro-1H-pyrazole-5-carboxylate (10 g. 34.7 mmol) in toluene (120 mL), copper (I) iodide (0.661 g, 3.47 mmol), 1, 10-phenanthroline (0.751 g, 4.16 mol), 3-thietanol (4.07 g, 45.1 mmol), potassium triphosphate (14.73 g, 69.4 mmol), and potassium carbonate (9.59 g, 69.4 mmol) were added at 40-45° C. under nitrogen atmosphere. The reaction mixture was stirred at 105-110° C. for 2-14 h. After completion of the reaction, the reaction mixture was cooled to 40-50° C. and filtered through a celite bed. The toluene layer was washed with brine solution (30 mL), dried over anhydrous sodium sulphate, and concentrated under reduced pressure to obtain a crude product. Isopropanol (10 mL) was added to the crude product and co-distilled off completely under reduced pressure. The residue obtained was dissolved in isopropanol (50 mL) at 40-45° C. and slowly cooled to 5-10° C. to obtain a solid. The solid obtained was filtered, washed with isopropanol (10 mL), and dried under reduced pressure to obtain pure ethyl 1-(3-chloropyridin-2-yl)-3-(thietan-3-yloxy)-4,5-dihydro-1H-pyrazole-5-carboxylate (8.5 g, 71.7% yield).

### Process-7: Ethyl 1-(3-chloropyridin-2-yl)-3-(thietan-3-yloxy)-4,5-dihydro-1H-pyrazole-5-carboxylate Using Anhydrous CuI as a Catalyst and Cs₂CO₃ as a Base

To a stirred solution of ethyl 3-chloro-1-(3-chloropyridin-2-yl)-4,5-dihydro-1H-pyrazole-5-carboxylate (20 g, 69.4 mmol) in toluene (240 mL), copper (I) iodide (1.32 g, 6.94 mmol), 1,10-phenanthroline (1.5 g. 8.33 mol), 3-thietanol (9.4 g, 104 mmol), cesium carbonate (45.2 g, 139 mmol) and potassium carbonate (19.19 g, 139 mmol) were added at 40-45° C. under nitrogen atmosphere. The reaction mixture was stirred at 105-110° ° C. for 2-9 h. After completion of the reaction, the reaction mixture was cooled to 40-50° C. and filtered through a celite bed. The toluene layer was washed with brine solution (60 mL), dried over anhydrous sodium sulphate and concentrated under reduced pressure to obtain crude ethyl 1-(3-chloropyridin-2-yl)-3-(thietan-3-yloxy)-4,5-dihydro-1H-pyrazole-5-carboxylate (15.76 g, 46.1 mmol, 66.4% yield)

### Example 3: Preparation of Ethyl 1-(3-chloropyridin-2-yl)-3-((1,1-dioxidothietan-3-yl)oxy)-1H-pyrazole-5-carboxylate

### Step 3: Process-1: Ethyl 1-(3-chloropyridin-2-yl)-3-((1,1-dioxidothietan-3-yl)oxy)-1H-pyrazole-5-carboxylate

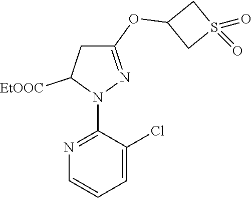 [SMILES: CCOC(=O)C1CC(OC2CS(=O)(=O)C2)=NN1c1ncccc1Cl]

To a stirred solution of ethyl 1-(3-chloropyridin-2-yl)-3-(thietan-3-yloxy)-4,5-dihydro-1H-pyrazole-5-carboxylate (100 g, 0.29 mol) in ethyl acetate (300 mL), powdered potassium permanganate (102 g, 0.64 mol) was charged at 25-30° ° C. The reaction mixture was cooled to 0-5° C., and glacial acetic acid (200 mL) was added drop wise during 1.5-2 h while maintaining the reaction temperature between 15 and 20° C. The reaction mixture was then stirred for 0.5-1 h at 25-30° C. followed by the addition of ethyl acetate (1200 mL) and 10% aqueous sulfuric acid solution (1000 mL) at the same temperature. The emulsion obtained was filtered through a celite bed and washed with ethyl acetate (200 mL). The ethyl acetate layer was separated, washed with 10% aqueous sulfuric acid solution (200 mL) and brine solution (200 mL), dried over anhydrous sodium sulphate and concentrated under reduced pressure to obtain ethyl 1-(3-chloropyridin-2-yl)-3-((1,1-dioxidothietan-3-yl)oxy)-1H-pyrazole-5-carboxylate (101 g, 93% yield).

¹H NMR (DMSO-d₆, 400 MHz): 8.54-8.52 (dd, J=1.2 Hz, 1H), 8.23-8.20 (dd, J=1.6 Hz, 1H), 7.66-7.63 (dd, J=4.8 Hz, 1H), 6.74 (s, 1H), 5.28 (m, 1H), 4.74 (m, 2H), 4.27 (m, 2H), 4.12 (q, 2H), 1.05 (t, 3H); MS: m/ [symbol] =372.20 [M+H]

### Process-2: Ethyl 1-(3-chloropyridin-2-yl)-3-((1,1-dioxidothietan-3-yl)oxy)-1H-pyrazole-5-carboxylate

To a mixture of ethyl acetate (1920 ml), ethyl 1-(3-chloropyridin-2-yl)-3-(thietan-3-yloxy)-4,5-dihydro-1H-pyrazole-5-carboxylate (48 g, 116 mmol), potassium permanganate lot-1 (55.2 g, 349 mmol) and manganese (IV) oxide lot-1 (202 g, 2328 mmol), 2,2,2-trifluoroacetic acid (17.83 ml, 233 mmol) was added drop wise maintaining the reaction temperature below 35° C. The reaction mixture was cooled to room temperature and stirred further for 12 h. Potassium permanganate lot-2 (23.6 g) and manganese (IV) oxide lot-2 (64.4 g) were added at 20-30° C. in one portion followed by dropwise addition of 2,2,2-trifluoroacetic acid (19 ml). After completion of the reaction, the reaction mixture was filtered through celite bed. The filtrate was washed with water and concentrated to obtain the crude product (36 g, 86%).

### Process-3: Ethyl 1-(3-chloropyridin-2-yl)-3-(thietan-3-yloxy)-1H-pyrazole-5-carboxylate

Ethyl 1-(3-chloropyridin-2-yl)-3-(thietan-3-yloxy)-4,5-dihydro-1H-pyrazole-5-carboxylate (0.20 g, 0.585 mmol) and activated MnO₂ were added to THF:EtOAc (1:5) at 25-30° C. TFA (4.51 μl, 0.059 mmol) was added and the reaction mixture was stirred at 25-50° C. for 8 h. After completion of the reaction, the reaction mixture was filtered through a celite bed. The filtrate was concentrated and purified by column chromatography to obtain pure ethyl 1-(3-chloropyridin-2-yl)-3-(thietan-3-yloxy)-1H-pyrazole-5-carboxylate (120 mg, 60%).

¹H-NMR (400 MHZ, DMSO-d6) δ 8.54-8.51 (m, 1H), 8.24-8.04 (m, 1H), 7.85-7.62 (m, 1H), 6.73-6.60 (m, 1H), 5.60-5.41 (m, 1H), 4.14-3.99 (m, 2H), 3.53-3.37 (m, 4H), 1.00-1.11 (3H); MS: m/ [symbol] =339.95 [M+H]

### Process-4: Ethyl 1-(3-chloropyridin-2-yl)-3-((1,1-dioxidothietan-3-yl)oxy)-1H-pyrazole-5-carboxylate

To a stirred solution of ethyl 1-(3-chloropyridin-2-yl)-3-(thietan-3-yloxy)-4,5-dihydro-1H-pyrazole-5-carboxylate (10 g, 29.3 mmol) in ethyl acetate (50 mL), TFA (7.34 g, 64.4 mmol) and powdered potassium permanganate (10.17 g, 64.4 mol) were added maintaining the temperature below 65° C. The reaction mixture was stirred for 1-3 h at 25-30° ° C. followed by the addition of ethyl acetate (100 mL) and quenching by addition of a 10% aqueous hydrochloric acid solution (100 mL) at the same temperature. Ethyl acetate layer was separated, washed with 10% aqueous hydrochloric acid solution (40 mL) and brine solution (40 mL), dried over anhydrous sodium sulphate and concentrated under reduced pressure to obtain crude ethyl 1-(3-chloropyridin-2-yl)-3-((1,1-dioxidothietan-3-yl)oxy)-1H-pyrazole-5-carboxylate (10.0 g, 92% yield).

### Process-5: Ethyl 1-(3-chloropyridin-2-yl)-3-((1,1-dioxidothietan-3-yl)oxy)-1H-pyrazole-5-carboxylate

To a stirred solution of ethyl 1-(3-chloropyridin-2-yl)-3-(thietan-3-yloxy)-4,5-dihydro-1H-pyrazole-5-carboxylate (100 g, 0.29 mol) in AcOH (500 mL), powdered potassium permanganate (79 g, 0.497 mol) was added maintaining the temperature below 65° C. The reaction mixture was stirred for 0.5-1 h at 25-30° C. followed by the addition of ethyl acetate (1200 mL) and quenching by addition of 10% aqueous hydrochloric acid solution (1000 mL) at the same temperature. The emulsion obtained was filtered through a celite bed and washed with ethyl acetate (200 mL). The ethyl acetate layer was separated, washed with 10% aqueous hydrochloric acid solution (200 mL) and brine solution (200 mL), dried over anhydrous sodium sulphate and concentrated under reduced pressure to obtain crude ethyl 1-(3-chloropyridin-2-yl)-3-((1,1-dioxidothietan-3-yl)oxy)-1H-pyrazole-5-carboxylate (101 g, 93% yield).

### Example 4: Preparation of 1-(3-chloropyridin-2-yl)-3-((1,1-dioxidothietan-3-yl) oxy)-1H-pyrazole-5-carboxylic Acid

### Step 4: Process-1: 1-(3-Chloropyridin-2-yl)-3-((1,1-dioxidothietan-3-yl) oxy)-1H-pyrazole-5-carboxylic Acid

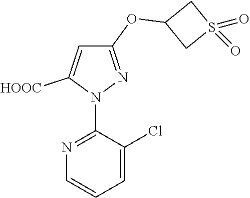 [SMILES: O=C(O)c1cc(OC2CS(=O)(=O)C2)nn1-c1ncccc1Cl]

To a stirred solution of ethyl 1-(3-chloropyridin-2-yl)-3-((1,1-dioxidothietan-3-yl)oxy)-1H-pyrazole-5-carboxylate (218 g, 0.44 mol) in glacial acetic acid (545 mL), a 20% aqueous sulfuric acid solution (1526 mL) was charged at 25-30° C. The reaction mixture was heated for 12 h at 95-100° C. under mild nitrogen gas bubbling, and the volatiles were distilled off by use of a downward distillation apparatus. The reaction mixture was quenched by the addition of water (1500 mL) during 0.5 h at 80-85° C. and allowed to cool for 2-3 h at 5-10° ° C. The solid obtained was filtered, washed with water (500 mL) and followed by ethyl acetate (200 mL) to obtain 1-(3-chloropyridin-2-yl)-3-((1,1-dioxidothietan-3-yl) oxy)-1H-pyrazole-5-carboxylic acid (141.5 g, 93% yield).

¹H NMR (DMSO-d₆, 400 MHz): 13.70 (br s, 1H), 8.52-8.50 (dd, J=1.6 Hz, 1H), 8.20-8.17 (dd, J=1.6 Hz, 1H), 7.63-7.60 (dd, J=4.8 Hz, 1H), 6.64 (s, 1H), 5.26 (m, 1H), 4.72 (m, 2H), 4.26 (m, 2H); MS: m/ [symbol] =342.25 [M−H]

### Process-2: 1-(3-chloropyridin-2-yl)-3-((1,1-dioxidothietan-3-yl) oxy)-1H-pyrazole-5-carboxylic Acid

A solution of ethyl 1-(3-chloropyridin-2-yl)-3-((1,1-dioxidothietan-3-yl)oxy)-1H-pyrazole-5-carboxylate (48 g, 0.129 mol) in 50% aqueous sulfuric acid solution (480 mL) was heated for 15-20 h at 105-115° C. After completion of the reaction, the reaction mixture was quenched by addition of water (480 mL). The solid obtained was filtered and washed with water (240 mL) to obtain 1-(3-chloropyridin-2-yl)-3-((1,1-dioxidothietan-3-yl) oxy)-1H-pyrazole-5-carboxylic acid (27 g, 60% yield).

### Process-3: 1-(3-chloropyridin-2-yl)-3-((1,1-dioxidothietan-3-yl) oxy)-1H-pyrazole-5-carboxylic Acid

A solution of ethyl 1-(3-chloropyridin-2-yl)-3-((1,1-dioxidothietan-3-yl)oxy)-1H-pyrazole-5-carboxylate (26 g, 0.069 mol) in 50% aqueous sulfuric acid solution (130 mL) was heated for 7-10 h at 105-110° C. under mild nitrogen gas bubbling. The volatiles were distilled via a downward distillation apparatus. The reaction mixture was quenched by addition of water (260 mL) during 0.5 h at 25-30° C. and allowed to cool for 2-3 h at 5-10° ° C. The solid obtained was filtered and washed with water (260 mL) to obtain 1-(3-chloropyridin-2-yl)-3-((1,1-dioxidothietan-3-yl) oxy)-1H-pyrazole-5-carboxylic acid (15 g, 62% yield).

### Process-4: 1-(3-chloropyridin-2-yl)-3-((1,1-dioxidothietan-3-yl) oxy)-1H-pyrazole-5-carboxylic Acid

To a stirred solution of ethyl 1-(3-chloropyridin-2-yl)-3-((1,1-dioxidothietan-3-yl)oxy)-1H-pyrazole-5-carboxylate (20 g, 0.53 mol) in glacial acetic acid (50 mL), 4N-6N hydrochloric acid solution (100 mL) was added at 25-30° ° C. The reaction mixture was heated for 6-8 h at 105-110° C. under mild nitrogen gas bubbling. The volatiles were distilled via a downward distillation apparatus. The reaction mixture was quenched by addition of water (1526 mL) during 0.5 h at 80-85° C. and allowed to stir for 2-3 h at 25-30° C. The solid obtained was filtered and washed with water (300 mL) to obtain 1-(3-chloropyridin-2-yl)-3-((1,1-dioxidothietan-3-yl) oxy)-1H-pyrazole-5-carboxylic acid (13.8 g, 75% yield).

### Example 5: Preparation of 6-chloro-2-(1-(3-chloropyridin-2-yl)-3-((1,1-dioxidothietan-3-yl)oxy)-1H-pyrazol-5-yl)-8-methyl-4H-benzo[d][1,3]oxazin-4-one

### Step 5: Process-1: 6-Chloro-2-(1-(3-chloropyridin-2-yl)-3-((1,1-dioxidothietan-3-yl)oxy)-1H-pyrazol-5-yl)-8-methyl-4H-benzo[d][1,3]oxazin-4-one

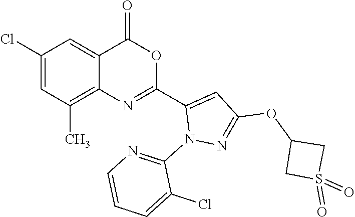 [SMILES: Cc1cc(Cl)cc2c(=O)oc(-c3cc(OC4CS(=O)(=O)C4)nn3-c3ncccc3Cl)nc12]

To a stirred suspension of 1-(3-chloropyridin-2-yl)-3-((1,1-dioxidothietan-3-yl)oxy)-1H-pyrazole-5-carboxylic acid (197.0 g, 0.57 mol) in acetonitrile (lot-1, 591 mL), pyridine (lot-1, 231 mL, 2.86 mol) was added at 25-30° ° C. The reaction mixture was cooled to 0-10° C., and methane sulfonyl chloride (lot-1, 66.5 mL, 0.86 mol) was added drop wise at the same temperature. In another reactor, to a stirred suspension of 2-amino-5-chloro-3-methylbenzoic acid (111.7 g, 0.60 mol) in acetonitrile (lot-2, 985 mL), pyridine (lot-2, 231 mL, 2.86 mol) was charged at 25-30° C. This reaction mixture was added to the above cooled solution at 0-10° C., and methane sulfonyl chloride (lot-2, 66.5 mL, 0.86 mol) was added drop wise at the same temperature. The resulting reaction mixture was stirred for 10-12 h at 25-30° C., cooled for 2-3 h at 0-5° C. and filtered. The filter cake obtained was suspended under stirring in water (lot-1, 1970 mL) and separated by filtration. The wet cake was washed again with water (lot-2, 197 mL) and dried under reduced pressure obtain 6-chloro-2-(1-(3-chloropyridin-2-yl)-3-((1,1-dioxidothietan-3-yl)oxy)-1H-pyrazol-5-yl)-8- to methyl-4H-benzo[d][1,3]oxazin-4-one (237 g, 83% yield).

¹H NMR (DMSO-d₆, 400 MHz): 8.58-8.57 (dd, J=1.6 Hz, 1H), 8.29-8.27 (dd, J=1.6 Hz, 1H), 7.87-7.86 (d, 1H), 7.74 (m, 1H), 7.69 (m, 1H), 6.92 (s, 1H), 5.34 (m, 1H), 4.77 (m, 2H), 4.30 (m, 2H), 1.69 (s, 3H); MS: m/ [symbol] =493.35 [M+H]

### Process-2: 6-Chloro-2-(1-(3-chloropyridin-2-yl)-3-((1,1-dioxidothietan-3-yl)oxy)-1H-pyrazol-5-yl)-8-methyl-4H-benzo[d][1,3]oxazin-4-one

A stirred suspension of 1-(3-chloropyridin-2-yl)-3-((1,1-dioxidothietan-3-yl) oxy)-1H-pyrazole-5-carboxylic acid (100.0 g, 0.284 mol) and 2-amino-5-chloro-3-methylbenzoic acid (53.3, 0.284 mol) in acetonitrile (800 mL) was cooled to 0-10° C. and pyridine (175 mL, 2.614 mol) was added at 0-10° C. Methane sulfonyl chloride (63.2 mL, 0.812 mol) was added drop wise at 0-10° C. The resulting reaction mixture was stirred for 1-2 h at 25-30° C., cooled for 3-6 h at 0-5° C. and filtered. The filter cake obtained was suspended under stirring in water (lot1 1000 mL) and filtered. The wet cake was washed with water (lot2, 250 mL) and dried under reduced pressure to obtain 6-chloro-2-(1-(3-chloropyridin-2-yl)-3-((1,1-dioxidothietan-3-yl)oxy)-1H-pyrazol-5-yl)-8-methyl-4H-benzo[d][1,3]oxazin-4-one (121 g, 91% yield).

### Process-3: 6-Chloro-2-(1-(3-chloropyridin-2-yl)-3-((1,1-dioxidothietan-3-yl)oxy)-1H-pyrazol-5-yl)-8-methyl-4H-benzo[d][1,3]oxazin-4-one

To a stirred suspension of 1-(3-chloropyridin-2-yl)-3-((1,1-dioxidothietan-3-yl) oxy)-1H-pyrazole-5-carboxylic acid (5.0 g, 0.014 mol) in dichloromethane (lot-1, 10 mL), pyridine (lot-1, 5.75 g, 0.072 mol) was added at 25-30° C. The reaction mixture was cooled to 0-10° C. and methane sulfonyl chloride (lot-1, 2.5 g, 0.021 mol) was added drop wise at the same temperature. In another reactor, to a stirred suspension of 2-amino-5-chloro-3-methylbenzoic acid (lot-1, 2.7 g, 0.014 mol) in dichloromethane (lot-2, 10 mL), pyridine (lot-2, 5.75 g, 0.072 mol) was added at 25-30° C. This reaction mixture was added to the above cooled solution at 0-10° C. and methane sulfonyl chloride (lot-2, 2.5 g, 0.021 mol) was added drop wise at the same temperature. The resultant reaction mixture was stirred for 10-15 h at 25-30° ° C., cooled for 2-3 h at 0-5° C. and filtered. The filter cake obtained was suspended under stirring in water (lot1, 50 mL), and filtered. The wet cake was washed with water (lot-2, and lot-3 25 mL and 25 mL) and dried under reduced pressure for to obtain 6-chloro-2-(1-(3-chloropyridin-2-yl)-3-((1,1-dioxidothietan-3-yl)oxy)-1H-pyrazol-5-yl)-8-methyl-4H-benzo[d][1,3]oxazin-4-one (5 g, 69% yield).

### Process-4: 6-Chloro-2-(1-(3-chloropyridin-2-yl)-3-((1,1-dioxidothietan-3-yl)oxy)-1H-pyrazol-5-yl)-8-methyl-4H-benzo[d][1,3]oxazin-4-one

To a stirred suspension of 1-(3-chloropyridin-2-yl)-3-((1,1-dioxidothietan-3-yl) oxy)-1H-pyrazole-5-carboxylic acid (5.0 g, 0.014 mol) in dichloroethane (lot-1, 12.5 mL), pyridine (lot-1, 5.75 g, 0.072 mol) was added at 25-30° C. The reaction mixture was cooled to 0-10° C. and methane sulfonyl chloride (lot-1, 2.5 g, 0.021 mol) was added drop wise at the same temperature. In another reactor, to a stirred suspension of 2-amino-5-chloro-3-methylbenzoic acid (lot-1, 2.7 g, 0.014 mol) in dichloroethane (lot-2, 12.5 mL), pyridine (lot-2, 5.75 g, 0.072 mol) was added at 25-30° C. This reaction mixture was added to the above cooled solution at 0-10° C. and methane sulfonyl chloride (lot-2, 2.5 g, 0.021 mol) was added drop wise at the same temperature. The resultant reaction mixture was stirred for 10-15 h at 25-30° ° C., cooled for 2-3 h at 0-5° C. and filtered. The filter cake obtained was suspended under stirring in water (lot-1, 50 mL), filtered, washed with water (lot2 and lot 3, 25 mL and 25 mL) and dried under reduced pressure to obtain 6-chloro-2-(1-(3-chloropyridin-2-yl)-3-((1,1-dioxidothietan-3-yl)oxy)-1H-pyrazol-5-yl)-8-methyl-4H-benzo[d][1,3]oxazin-4-one (4.2 g, 58% yield).

### Process-5: 6-chloro-2-(1-(3-chloropyridin-2-yl)-3-((1,1-dioxidothietan-3-yl)oxy)-1H-pyrazol-5-yl)-8-methyl-4H-benzo[d][1,3]oxazin-4-one

A stirred suspension of 1-(3-chloropyridin-2-yl)-3-((1,1-dioxidothietan-3-yl) oxy)-1H-pyrazole-5-carboxylic acid (25.0 g, 0.0727 mol) and 2-amino-5-chloro-3-methylbenzoic acid (13.5 g, 0.0727 mol) in N,N-dimethylformamide (75 mL) was cooled to 0-10° C. and ß-picoline (40.6 g, 0.436 mol) was added at 0-10° C. Methane sulfonyl chloride (25.0 g, 0.218 mol) was added drop wise at 0-10° C. The resultant reaction mixture was stirred for 6-12 h at 25-30° ° C. After completion of the reaction, the reaction mixture was poured slowly into pre-cooled water (375 mL) and stirred for 30-45 min at 20-30° C. The precipitated solid was filtered, washed with water (175 mL) and dried under reduced pressure to obtain pure 6-chloro-2-(1-(3-chloropyridin-2-yl)-3-((1,1-dioxidothietan-3-yl)oxy)-1H-pyrazol-5-yl)-8-methyl-4H-benzo[d][1,3]oxazin-4-one (21 g, 58% yield).

### Example 6: Preparation of N-(2-(tert-butylcarbamoyl)-4-chloro-6-methylphenyl)-1-(3-chloropyridin-2-yl)-3-((1,1-dioxidothietan-3-yl)oxy)-1H-pyrazole-5-carboxamide

### Step 6: Process-1: N-(2-(tert-butylcarbamoyl)-4-chloro-6-methylphenyl)-1-(3-chloropyridin-2-yl)-3-((1,1-dioxidothietan-3-yl)oxy)-1H-pyrazole-5-carboxamide

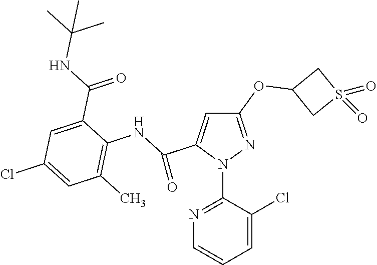 [SMILES: Cc1cc(Cl)cc(C(=O)NC(C)(C)C)c1NC(=O)c1cc(OC2CS(=O)(=O)C2)nn1-c1ncccc1Cl]

To a stirred suspension of 6-chloro-2-(1-(3-chloropyridin-2-yl)-3-((1,1-dioxidothietan-3-yl)oxy)-1H-pyrazol-5-yl)-8-methyl-4H-benzo[d][1,3]oxazin-4-one (57 g, 0.12 mol) in N,N-dimethylformamide (114 mL), tert-butyl amine (15.1 mL, 0.17 mol) was added drop wise during 30-40 min at 10-20° C. The reaction mixture was stirred for 6-8 h at 25-30° ° C. After completion of the reaction, excess tert-butyl amine was distilled off from the reaction mixture under reduced pressure, isopropanol was added (lot-1, 1140 mL), and the resulting mixture was stirred for 6-8 h at 25-30° C. The solid obtained was isolated by filtration, the wet cake obtained was suspended in isopropanol (lot-2, 114 mL) and filtered. The solid obtained was suspended again in acetone (lot-1, 285 mL) for 2-3 h at 55-60° C., cooled for 1-2 h at 25-30° C., isolated by filtration, washed with acetone (lot-2, 57 mL) and dried under reduced pressure to obtain N-(2-(tert-butylcarbamoyl)-4-chloro-6-methylphenyl)-1-(3-chloropyridin-2-yl)-3-((1,1-dioxidothietan-3-yl)oxy)-1H-pyrazole-5-carboxamide (57 g, 87% yield).

¹H NMR (DMSO-d₆, 400 MHz): 10.1 (s, 1H), 8.44-8.42 (dd, J=1.6 Hz, 1H), 8.11-8.09 (dd, J=1.6 Hz, 1H), 7.60 (br s, 1H), 7.55-7.52 (dd, J=4.8 Hz, 1H), 7.42-7.24 (dd, J=2.0 Hz, 2H), 6.78 (s, 1H), 5.29 (m, 1H), 4.72 (m, 2H), 4.26 (m, 2H), 2.13 (s, 3H), 1.24 (s, 9H); MS: m/ [symbol] =565.6 [M+H]

### Process-2: N-(2-(tert-butylcarbamoyl)-4-chloro-6-methylphenyl)-1-(3-chloropyridin-2-yl)-3-((1,1-dioxidothietan-3-yl)oxy)-1H-pyrazole-5-carboxamide

To a stirred suspension of 6-chloro-2-(1-(3-chloropyridin-2-yl)-3-((1,1-dioxidothietan-3-yl)oxy)-1H-pyrazol-5-yl)-8-methyl-4H-benzo[d][1,3]oxazin-4-one (5 g, 0.01 mol) in dimethyl sulfoxide (15 mL), tert-butyl amine (2.24 g, 0.0304 mol) was added drop wise over 30-40 min at 10-20° C. The reaction mixture was stirred for 3-6 h at 25-30° C. After completion of the reaction, the reaction mixture was poured slowly into pre-cooled water (75 ml) and stirred for 30-45 min at 20-30° C. The precipitate was filtered and washed with water (30 ml). This wet solid was taken in methanol (30 ml), heated to reflux for 1 h, cooled to 25-30° C. and stirred for 1-2 h at 25-30° C. The precipitate was filtered, washed with methanol (15 ml) and dried under reduced pressure to obtain N-(2-(tert-butylcarbamoyl)-4-chloro-6-methylphenyl)-1-(3-chloropyridin-2-yl)-3-((1,1-dioxidothietan-3-yl)oxy)-1H-pyrazole-5-carboxamide (3.7 g, 64% yield).

### Process-3: N-(2-(tert-butylcarbamoyl)-4-chloro-6-methylphenyl)-1-(3-chloropyridin-2-yl)-3-((1,1-dioxidothietan-3-yl)oxy)-1H-pyrazole-5-carboxamide

To a stirred suspension of 6-chloro-2-(1-(3-chloropyridin-2-yl)-3-((1,1-dioxidothietan-3-yl)oxy)-1H-pyrazol-5-yl)-8-methyl-4H-benzo[d][1,3]oxazin-4-one (2.5 g, 0.005 mol) in N,N-dimethyl formamide (6.25 mL), tert-butyl amine (0.92 g, 0.0126 mol) was added drop wise over 30-40 min at 10-20° C. The reaction mixture was stirred for 3-6 h at 25-30° ° C. After completion of the reaction, the reaction mixture was poured slowly into pre-cooled water (30 ml) and stirred for 30-45 min at 20-30° C. The precipitated solid was filtered and washed with water (15 ml). This wet solid was taken in methanol (15 ml), heated to reflux for 1 h, cooled to 25-30° C. and stirred for 1-2 h at 25-30° C. The precipitated solid was filtered, washed with methanol (7.5 ml) and dried under reduced pressure to obtain N-(2-(tert-butylcarbamoyl)-4-chloro-6-methylphenyl)-1-(3-chloropyridin-2-yl)-3-((1,1-dioxidothietan-3-yl)oxy)-1H-pyrazole-5-carboxamide (1.9 g, 66% yield).

### Process-4: N-(2-(tert-butylcarbamoyl)-4-chloro-6-methylphenyl)-1-(3-chloropyridin-2-yl)-3-((1,1-dioxidothietan-3-yl)oxy)-1H-pyrazole-5-carboxamide

To a stirred solution of 1-(3-chloropyridin-2-yl)-3-((1,1-dioxidothietan-3-yl)oxy)-1H-pyrazole-5-carboxylic acid (0.9 g, 2.62 mmol) in N,N-dimethylacetamide (10 ml), pyridine (0.633 ml, 7.85 mmol) was added at 0° C., followed by drop wise addition of and methanesulfonyl chloride (0.304 ml, 3.93 mmol) during 5 min and stirred for 10-15 min at 25-30° C. 2-Amino-N-(tert-butyl)-5-chloro-3-methylbenzamide (0.693 g, 2.88 mmol) was added to it and stirred at 55° C. for 16 h. After completion of the reaction, the reaction mixture was poured into ice water. The solid obtained was filtered and dried under reduced pressure to obtain the desired product N-(2-(tert-butylcarbamoyl)-4-chloro-6-methylphenyl)-1-(3-chloropyridin-2-yl)-3-((1,1-dioxidothietan-3-yl)oxy)-1H-pyrazole-5-carboxamide (0.70 g, 1.236 mmol, 47.2% yield)) as an off-white solid.

### Example-7: Preparation of Ethyl 2-(3-chloropyridin-2-yl)-5-oxo-2,5-dihydro-1H-pyrazole-3-carboxylate

To a solution of ethyl 2-(3-chloropyridin-2-yl)-5-oxopyrazolidine-3-carboxylate (10 g, 37.1 mmol) in DCM (200 ml), activated manganese dioxide (70.0 g, 806 mmol) was added at 20-23° C. under stirring. The reaction mixture was then stirred at 25° C. for 16 h. After completion of reaction, the reaction mixture was filtered through a celite bed. The filtrate was distilled out under reduced pressure to obtain crude material This crude material was charged into isopropanol (25 mL), stirred for 2 h and filtered to obtain pure ethyl 2-(3-chloropyridin-2-yl)-5-oxo-2,5-dihydro-1H-pyrazole-3-carboxylate (6.1 g, 22.79 mmol, 61.5% yield).

1H-NMR (400 MHZ, DMSO-d₆) δ 10.63 (s, 1H), 8.50 (dd, J=1.6 Hz, 1H), 8.17 (dd, J=1.6 Hz, 1H), 7.60 (dd, J=4.4 Hz, 1H), 6.33 (s, 1H), 4.11 (q. 2H), 1.05 (d, 3H); MS: m/ [symbol] =268.0 [M+H]

### Example-8: Preparation of Ethyl 1-(3-chloropyridin-2-yl)-3-(thietan-3-yloxy)-1H-pyrazole-5-carboxylate

Triphenylphosphine (1.5 eq), ethyl 1-(3-chloropyridin-2-yl)-3-hydroxy-1H-pyrazole-5-carboxylate (1.0 eq.) and thietan-3-ol (1.5 eq) was dissolved in tetrahydrofuran (10 ml) at 27° ° C. The reaction mixture was heated to 50° C. DIAD (1.5 eq.) was added drop wise, and the reaction was continued for 5-6 h. After completion of the reaction, the reaction was quenched by addition of water and extracted with ethyl acetate. The organic layer was dried over anhydrous sodium sulphate, filtered and concentrated under reduced pressure to obtain a crude product. The crude product was purified by column chromatography to obtain ethyl 1-(3-chloropyridin-2-yl)-3-(thietan-3-yloxy)-1H-pyrazole-5-carboxylate (55-60% yield)

1H-NMR (400 MHZ, CHLOROFORM-D) δ 8.49 (dd, J=4.8, 1.6 Hz, 1H), 7.88 (dd, J=7.9, 1.6 Hz, 1H), 7.40 (dd, J=7.9, 4.8 Hz, 1H), 6.40 (s, 1H), 5.64-5.56 (m, 1H), 4.19 (q. J=7.1 Hz, 2H), 3.59 (td, J=8.0, 1.8 Hz, 2H), 3.43-3.34 (m, 3H), 1.19 (t. J=7.1 Hz, 3H). MS: m/ [symbol] =340.0 [M+H]

### Example-9: Preparation of Ethyl 1-(3-chloropyridin-2-yl)-3-(thietan-3-yloxy)-1H-pyrazole-5-carboxylate

To a solution of ethyl 1-(3-chloropyridin-2-yl)-3-hydroxy-1H-pyrazole-5-carboxylate (1.0 e.) and thietan-3-ol (1.5 eq.) in toluene (20 ml) at 27° C., (2-hydroxybenzyl)diphenylphosphine oxide (10-25 mol %) and TFA (0.086 ml, 1.121 mmol) were added at 27° ° C. The reaction mixture was refluxed at 110-115° C. After completion of reaction, the reaction mixture was filtered through a celite bed. The filtrate obtained was concentrated under reduced pressure to obtain a crude product which was purified by column chromatography to afford ethyl 1-(3-chloropyridin-2-yl)-3-(thietan-3-yloxy)-1H-pyrazole-5-carboxylate.

### Example-10: Preparation of Ethyl 1-(3-chloropyridin-2-yl)-3-((1,1-dioxidothietan-3-yl)oxy)-4,5-dihydro-1H-pyrazole-5-carboxylate

To a solution of ethyl 1-(3-chloropyridin-2-yl)-3-(thietan-3-yloxy)-4,5-dihydro-1H-pyrazole-5-carboxylate (5.0 g, 14.63 mmol) in acetic acid (20 ml), hydrogen peroxide (4.19 ml, 117 mmol) was added slowly at 25-30° C., and the reaction mixture was stirred for 24 h at the same temperature. After completion of the reaction, the reaction mixture was quenched by addition of sodium bisulfite extracted with ethyl acetate and concentrated under reduced pressure to obtain ethyl 1-(3-chloropyridin-2-yl)-3-((1,1-dioxidothietan-3-yl)oxy)-4,5-dihydro-1H-pyrazole-5-carboxylate. LCMS [M+H]: 374.2 (50%).

### Example-11: Preparation of Ethyl 1-(3-chloropyridin-2-yl)-3-((1,1-dioxidothietan-3-yl)oxy)-1H-pyrazole-5-carboxylate

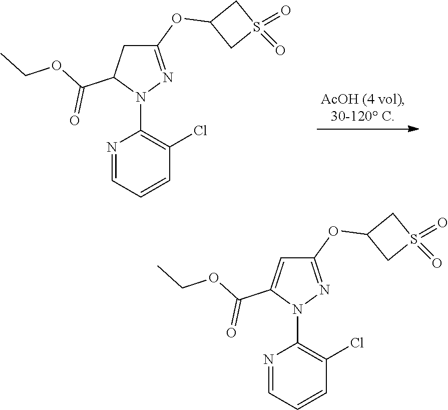 [SMILES: CCOC(=O)C1CC(OC2CS(=O)(=O)C2)=NN1c1ncccc1Cl ; CCOC(=O)c1cc(OC2CS(=O)(=O)C2)nn1-c1ncccc1Cl]

A solution of ethyl 1-(3-chloropyridin-2-yl)-3-((1,1-dioxidothietan-3-yl)oxy)-4,5-dihydro-1H-pyrazole-5-carboxylate (20 mg, 0.054 mmol) in acetic acid (0.8 ml) was stirred at 120° C. for 18 h. After completion of the reaction, the solvent was distilled. The reaction mass obtained was diluted with water, neutralized with sodium bicarbonate solution and extracted with ethyl acetate. The ethyl acetate layer was concentrated under reduced pressure to obtain a crude product ethyl 1-(3-chloropyridin-2-yl)-3-((1,1-dioxidothietan-3-yl)oxy)-1H-pyrazole-5-carboxylate. LCMS [M+H]: 372.00 (76.2%).

### Example 12

### Step-1: Synthesis of Ethyl 1-(3-chloropyridin-2-yl)-3-((methylsulfonyl)oxy)-4,5-dihydro-1H-pyrazole-5-carboxylate

To a solution of ethyl 2-(3-chloropyridin-2-yl)-5-oxopyrazolidine-3-carboxylate (100 g, 367 mmol) in dichloromethane (700 mL), triethylamine (179 mL, 1285 mmol) and methanesulfonyl chloride (42.9 mL, 551 mmol) were added at −10-0° ° C. The reaction mass was stirred at 0-5° C. for 3 h. After completion of the reaction, the reaction mass was washed with water (500 mL) and extracted with dichloromethane (250 mL). The combined organic layers were washed with 10% aq. NaHCO₃ (500 mL) and brine solution (500 mL), dried over anhydrous sodium sulphate, filtered and concentrated under reduced pressure to afford a crude product (111 g, 319 mmol, 87% yield). The crude product was dissolved in ethanol (218 mL) at 50-55° C. and gradually cooled to 25-30° C. The resulting precipitate was filtered, washed with ethanol (50 mL) and dried to afford ethyl 1-(3-chloropyridin-2-yl)-3-((methylsulfonyl)oxy)-4,5-dihydro-1H-pyrazole-5-carboxylate as a pale brown solid (79.85 g, 73% yield).

¹H NMR (DMSO-d₆, 400 MHz): δ 8.12-8.10 (dd, J=1.6 Hz, 1H), 7.84-7.82 (dd, J=1.6 Hz, 1H), 6.98-6.95 (dd, J=4.8 Hz, 1H), 5.28-5.23 (m, 1H), 4.11 (q, 2H), 3.67 (s, 3H), 3.54-3.47 (m, 1H), 3.20-3.16 (m, 1H), 1.14 (t, 3H), 3.31 (m, 1H), 2.93 (m, 1H), 1.15 (t, 3H) MS: m/z=348.0 [M+H]⁺

### Synthesis of Ethyl 1-(3-chloropyridin-2-yl)-3-(((trifluoromethyl)sulfonyl)oxy)-4,5-dihydro-1H-pyrazole-5-carboxylate

To a solution of ethyl 2-(3-chloropyridin-2-yl)-5-oxopyrazolidine-3-carboxylate (5.0 g, 18.54 mmol) in dichloromethane (50 mL), triethylamine (1.87 g, 18.54 mmol) and triflic anhydride (3.13 mL, 18.54 mmol) were added at 0-5° C. The reaction mass was stirred at 0-5° C. for 3 h and then temperature was raised to 25-30° C. and stirred at the same temperature for 20 h. After completion of the reaction, the reaction mixture was concentrated under reduced pressure and nitrogen atmosphere to obtain 1-(3-chloropyridin-2-yl)-3-(((trifluoromethyl)sulfonyl)oxy)-4,5-dihydro-1H-pyrazole-5-carboxylate as crude oil.

### Synthesis of Ethyl 1-(3-chloropyridin-2-yl)-3-(tosyloxy)-4,5-dihydro-1H-pyrazole-5-carboxylate

To a solution of ethyl 2-(3-chloropyridin-2-yl)-5-oxopyrazolidine-3-carboxylate (100 g, 371 mmol) in dichloromethane (250 mL), triethylamine (114 mL, 816 mmol) and a solution of para-toluene sulfonyl chloride (78 g, 408 mmol) in dichloromethane (250 mL) were added at 15-20° C. The reaction mass was stirred at 15-20° C. for 2-3 h. After completion of the reaction, the reaction mixture was washed with water (2×125 mL) and extracted with dichloromethane (2×50 mL). The combined organic layers were washed with 10% aq. NaHCO₃ (250 mL) and brine solution (250 mL), dried over anhydrous sodium sulphate, filtered and concentrated under reduced pressure to afford ethyl 1-(3-chloropyridin-2-yl)-3-(tosyloxy)-4,5-dihydro-1H-pyrazole-5-carboxylate (150 g, 95%).

¹H NMR (CDCl₃, 400 MHz): δ 7.98-7.97 (dd, J=1.6 Hz, 1H), 7.92-7.90 (d, J=8.0 Hz, 2H), 7.54-7.51 (dd, J=1.6 Hz, 1H), 7.33-7.31 (d, 2H), 6.77-9.74 (m, 1H), 5.22-5.17 (m, 1H), 4.14 (q, 2H), 3.35-3.27 (m, 1H), 3.12-3.06 (m, 1H), 2.41 (s, 3H), 1.16 (t, 3H).

MS: m/z=424.8 [M+H]⁺

### Step-2: Synthesis of Ethyl 1-(3-chloropyridin-2-yl)-3-(thietan-3-yloxy)-4,5-dihydro-1H-pyrazole-5-carboxylate

Process-1: To a solution of ethyl 1-(3-chloropyridin-2-yl)-3-((methylsulfonyl)oxy)-4,5-dihydro-1H-pyrazole-5-carboxylate (0.50 g, 1.436 mmol) in toluene (6 mL), thietan-3-ol (0.15 g, 1.725 mmol) and CuCl (0.043 g, 0.3 eq.) were added at 25-30° ° C. The reaction mixture was heated at 110° C. for 20 h. After completion of the reaction, the reaction mixture was filtered through a celite bed and washed with toluene (3 mL). The combined toluene layer were concentrated under reduced pressure to afford ethyl 1-(3-chloropyridin-2-yl)-3-(thietan-3-yloxy)-4,5-dihydro-1H-pyrazole-5-carboxylate as a pale brown solid (0.3 g, 51%).

Process-2: To a solution of ethyl 1-(3-chloropyridin-2-yl)-3-((methylsulfonyl)oxy)-4,5-dihydro-1H-pyrazole-5-carboxylate (0.5 g, 1.436 mmol) in chlorobenzene (2.5 mL), thietan-3-ol (0.17 g, 1.87 mmol) and DBU (0.28 mL, 1.87 mmol) were added at 25-30° C. The resulting reaction mixture was heated at 130° C. for 0.5 h. After completion of the reaction, water (5 mL) was added at 25-30° C. and extracted with EtOAc (2×20 mL). The combined organic layers were dried over anhydrous sodium sulphate, filtered and concentrated under reduced pressure to afford ethyl 1-(3-chloropyridin-2-yl)-3-(thietan-3-yloxy)-4,5-dihydro-1H-pyrazole-5-carboxylate as thick oil, which was purified in isopropanol (2 mL) to afford ethyl 1-(3-chloropyridin-2-yl)-3-(thietan-3-yloxy)-4,5-dihydro-1H-pyrazole-5-carboxylate as a pale brown solid (0.3 g, 61%).

### Example-13

### Step-1: Synthesis of Ethyl 1-(3-chloropyridin-2-yl)-3-((1,1-dioxidothietan-3-yl)oxy)-1H-pyrazole-5-carboxylate

To a solution of ethyl 1-(3-chloropyridin-2-yl)-3-(thietan-3-yloxy)-4,5-dihydro-1H-pyrazole-5-carboxylate (50 g, 146 mmol) in dichloroethane (250 mL), nitric acid (70% solution, 8.72 mL, 146 mmol) was added dropwise at 20-30° ° C. The reaction mass was stirred at 20-30° C. for 5-6 h. After completion of the reaction, water (200 mL) was added under stirring at 20-30° C. and layers were separated. The organic layer was washed with water (200 mL) and concentrated under reduced pressure to afford ethyl 1-(3-chloropyridin-2-yl)-3-(thietan-3-yloxy)-1H-pyrazole-5-carboxylate as off-white solid (49.07 g, 99%). This crude was taken as such for the next step.

### Step-2: Synthesis of Ethyl 1-(3-chloropyridin-2-yl)-3-((1,1-dioxidothietan-3-yl)oxy)-1H-pyrazole-5-carboxylate

To a solution of ethyl 1-(3-chloropyridin-2-yl)-3-(thietan-3-yloxy)-1H-pyrazole-5-carboxylate (49 g, 144 mmol) in acetic acid (122.5 mL), sodium tungstate (0.42 g, 1.442 mmol) was added at 20-30° C. To this mixture, hydrogen peroxide (30% aq. solution, 36.8 mL, 361 mmol) was added at 20-30° C. during 1 h, and the reaction continued at 20-30° C. for 5-6 h. After completion of the reaction, water (245 mL) and ethyl acetate (123 mL) were added under stirring at 20-30°. The organic layer was washed with 5% aq. Na₂S₂O₅ (200 mL) and water (200 mL) and concentrated under reduced pressure to afford ethyl 1-(3-chloropyridin-2-yl)-3-((1,1-dioxidothietan-3-yl)oxy)-1H-pyrazole-5-carboxylate as an off-white solid. (50.5 g, 94%)

¹H NMR (DMSO-d6, 400 MHz): δ 8.54-8.52 (dd, J=1.2 Hz, 1H), 8.23-8.20 (dd, J=1.6 Hz, 1H), 7.66-7.63 (dd, J=4.8 Hz, 1H), 6.74 (s, 1H), 5.28 (m, 1H), 4.74 (m, 2H), 4.27 (m, 2H), 4.12 (q, 2H), 1.05 (t, 3H).

MS: m/z=372.20 [M+H]⁺

1. A process for preparing a compound of formula (IV), 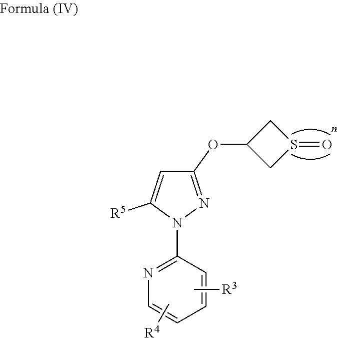
wherein,
R′ is selected from the group consisting of COOH and COX,
X represents halogen selected from Cl or Br;
R³ is selected from the group consisting of halogen, C₁-C₄ alkyl and C₁-C₄ haloalkyl;
R⁴ is selected from the group consisting of hydrogen, halogen, C₁-C₄ alkyl and C₁-C₄ haloalkyl;
n represents an integer selected from 0 to 2; or salts thereof;
comprising the steps of:
a) obtaining a compound of formula (VII) from a compound of formula (VIII); 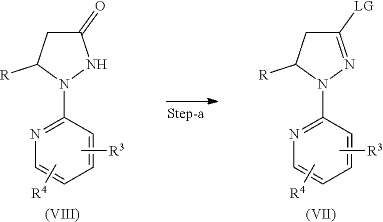
wherein, LG is selected from the group consisting of halogen, OMs, OTf and OTs; R is selected from the group consisting of CX₃, CN and COOR^{c}, R^{c} represents C₁-C₄ alkyl; X, R³ and R⁴ are as defined herein above;
b) reacting the compound of formula (VII) with a compound of formula (X) to obtain a compound of formula (VI); 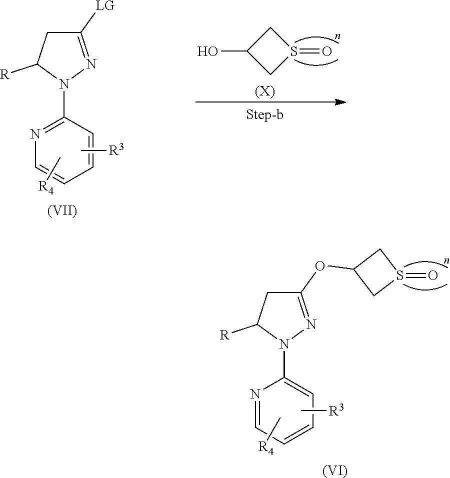
wherein, n, R, R³, R⁴ and LG are as defined herein above;
c) oxidizing the compound of formula (VI) by the use of a suitable oxidizing agent and a suitable acid to obtain a compound of formula (V); 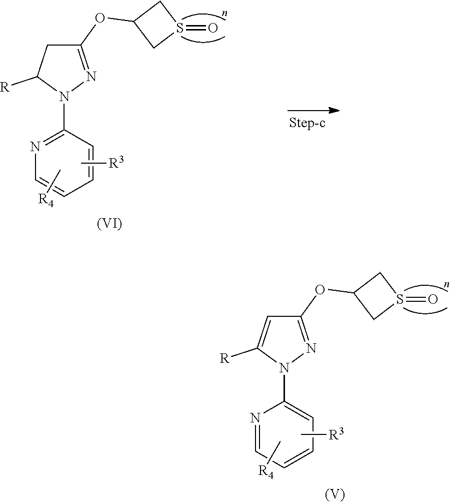
wherein, n, R, R³ and R⁴ are as defined herein above;
d) converting the compound of formula (V) into a compound of formula (IV); 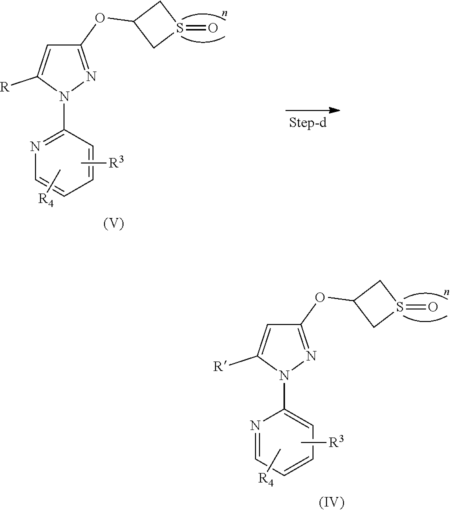
wherein, R′ represents COOH or COX, and n, R, R³ and R⁴ are as defined herein above.

2. The process as claimed in claim 1, wherein said process further comprises the step of: reacting the compound of formula (IV) with a compound of formula (III) and a suitable amine of formula (R^{a}R^{b}NH) or with a compound of formula (IIIa) to obtain a compound of formula (I); 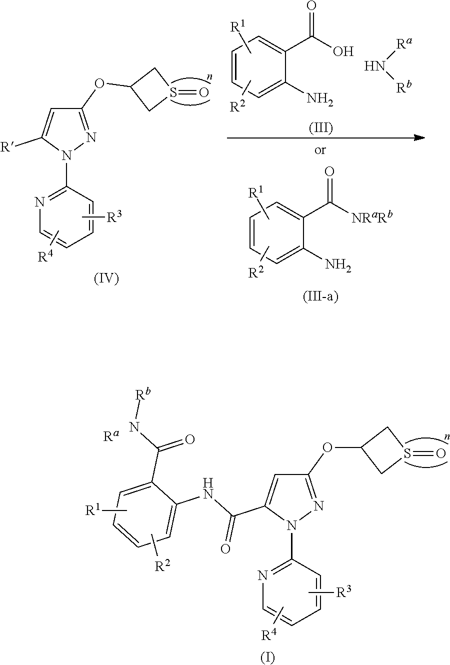
wherein,
R^{a} and R^{b} are independently selected from the group consisting of hydrogen, C₁-C₆ alkyl, C₃-C₆ cycloalkyl and C₃-C₆ cycloalkyl-C₁-C₄ alkyl; wherein R^{a} and R^{b} optionally substituted with one or more halogen;
R′ is selected from the group consisting of COOH or COX;
X is halogen;
R¹ and R² are independently selected from the group consisting of hydrogen, halogen, cyano, C₁-C₆ alkyl, C₁-C₄ haloalkyl, and C₃-C₆ cycloalkyl;
R³ is selected from the group consisting of halogen, C₁-C₄ alkyl and C₁-C₄ haloalkyl;
R⁴ is selected from the group consisting of hydrogen, halogen, C₁-C₄ alkyl and C₁-C₄ haloalkyl;
n represents an integer selected from 0 to 2; and R′, is as defined in claim 1.

3. The process as claimed in claim 2, wherein said process is carried out in the presence of a suitable halogenating agent and a suitable base.

4. The process step c) as claimed in claim 1 for the preparation of compound of formula (V) wherein n=1 or 2, is obtained by using the process comprising of:
c) converting the compound of formula (VI) wherein n=0 into a compound of formula (VI) wherein n=1 or 2, using a suitable oxidizing agent, which on further reaction with a suitable acid provides a compound of formula (V) 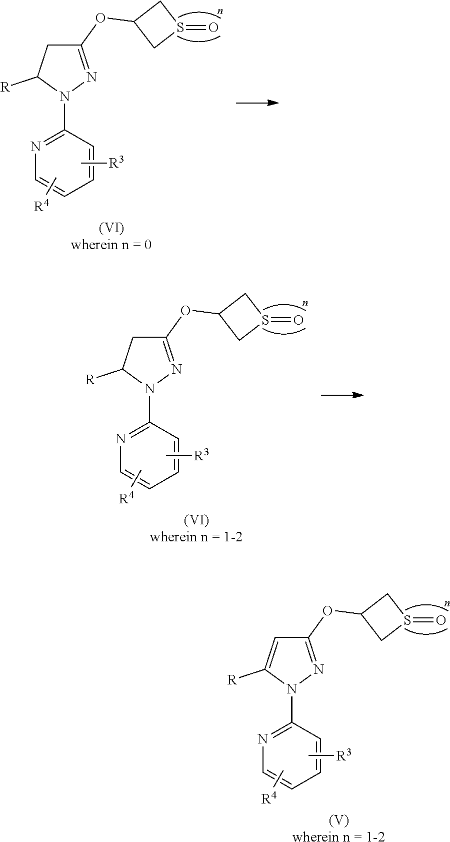
wherein, R, R³ and R⁴ are as defined in claim 1;
or
c) converting the compound of formula (VI) into a compound of formula (VI) wherein n=0 reacting with suitable acid, which on further oxidation using a suitable oxidizing agent to obtain a compound of formula (V); 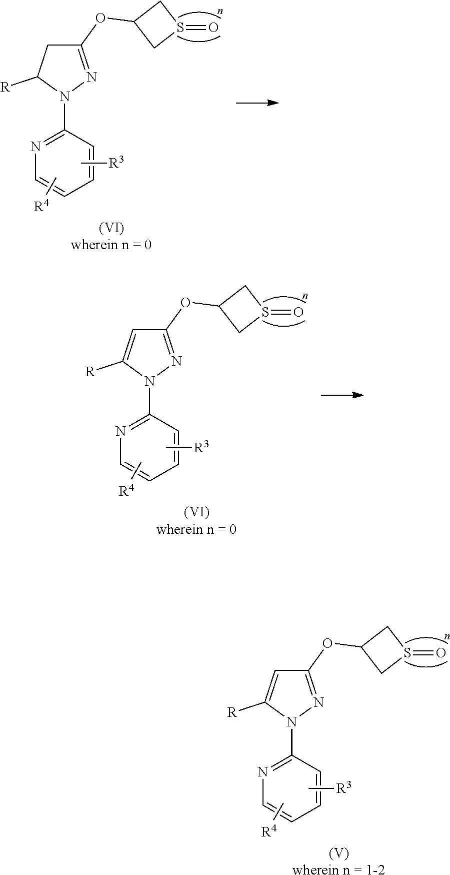
wherein, R, R³ and R⁴ are as defined in claim 1.

5. The process as claimed in claim 1, wherein step-d) is carried out by using the following process,
d-1) converting a compound of formula (V) into a compound of formula (IV) using suitable hydrolyzing agents; 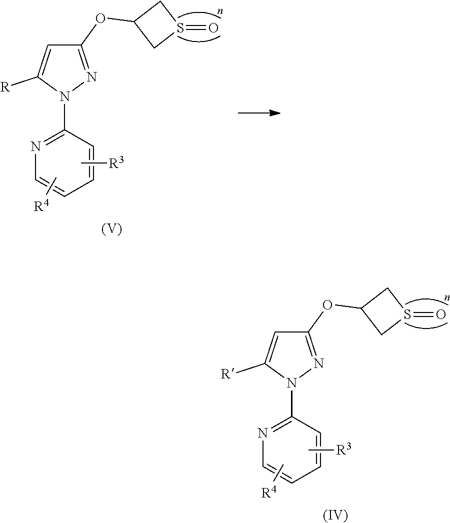
wherein, R′ represents COOH, and n, R, R³ and R⁴ are as defined in claim 1;
d-2) converting the compound of formula (IV) wherein R′ represents COOH to a compound of formula (IV) wherein R′ represents COX, using a suitable halogenating agent; 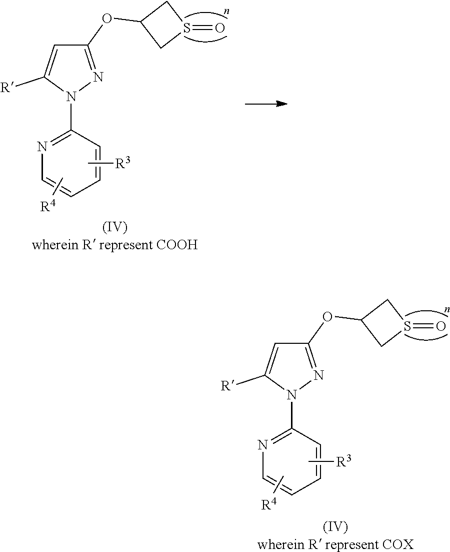
wherein n, X, R, R³ and R⁴ are as defined in claim 1.

6. A process for the preparation of compound of formula (IV) as claimed in claim 1, comprising the steps of:
a) oxidizing a compound of formula (VIII) using a suitable oxidizing agent or a suitable acid to obtain a compound of formula (IX); 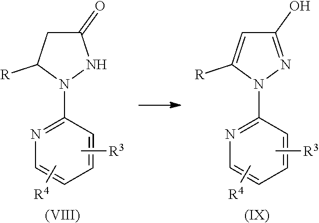
wherein, R is selected from CX₃, CN or COOR^{c}, R^{c} represents C₁-C₄ alkyl; X, R³ and R⁴ are as defined in claim 1;
b) reacting the compound of formula (IX) with a compound of formula (X) to obtain a compound of formula (V); 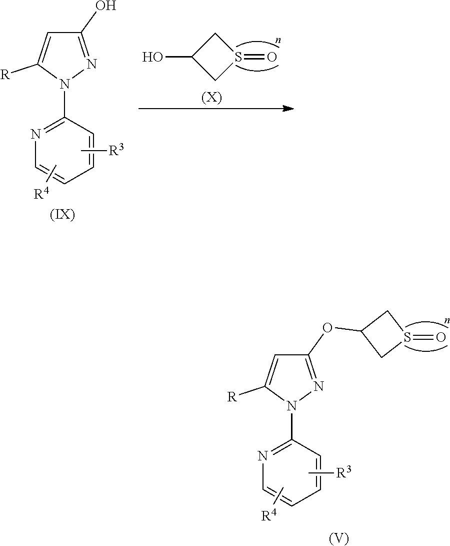
wherein n R, R³, R⁴ are as defined in claim 1;
c) converting the compound of formula (V) to a compound of formula (IV); 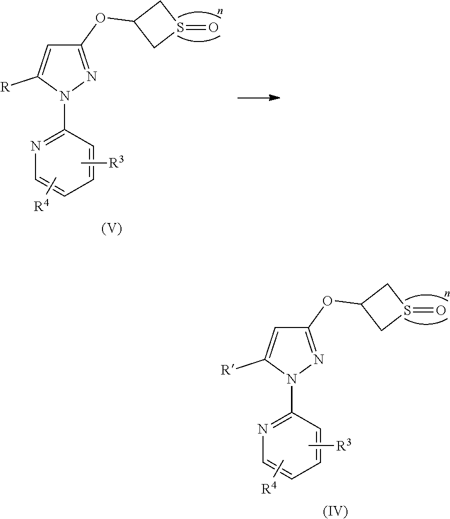
wherein, R′ represent COOH or COX; n, X, R, R³ and R⁴ are as defined in claim 1.

7. A process for the synthesis of compound of formula (I), 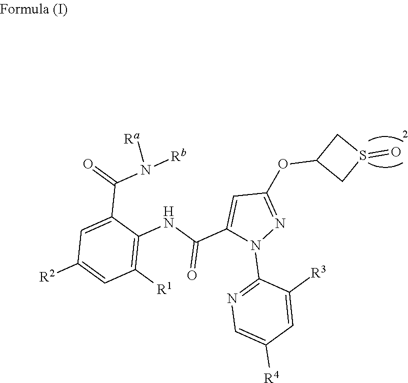
wherein,
R^{a} and R^{b} are independently selected from the group consisting of hydrogen, C₁-C₆ alkyl, C₃-C₆ cycloalkyl and C₃-C₆ cycloalkyl-C₁-C₄ alkyl; wherein R^{a} and R^{b} optionally substituted with one or more halogen;
R¹ and R² are independently selected from the group consisting of hydrogen, halogen, cyano, C₁-C₆ alkyl, C₁-C₄ haloalkyl, and C₃-C₆ cycloalkyl;
R³ is selected from the group consisting of halogen, C₁-C₄ alkyl and C₁-C₄ haloalkyl;
R⁴ is selected from the group consisting of hydrogen, halogen, C₁-C₄ alkyl and C₁-C₄ haloalkyl;
n represents an integer 2; or salts or N-oxides thereof;
from the compound of formula (IV) as prepared in claim 1,
comprising the steps of:
a) obtaining a compound of formula (VIIa) from a compound of formula (VIIIa); 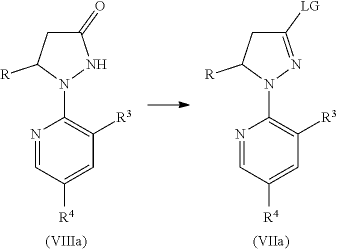
wherein, LG is selected from the group consisting of halogen, OMs, OTf and OTs; R is selected from the group consisting of CX₃, CN and COOR^{c}, R^{c} represents C₁-C₄ alkyl; X represents halogen; R³ and R⁴ are as defined herein above;
b) reacting the compound of formula (VIIa) with a compound of formula (X) to obtain a compound of formula (VIa), in the presence of suitable base and a suitable solvent 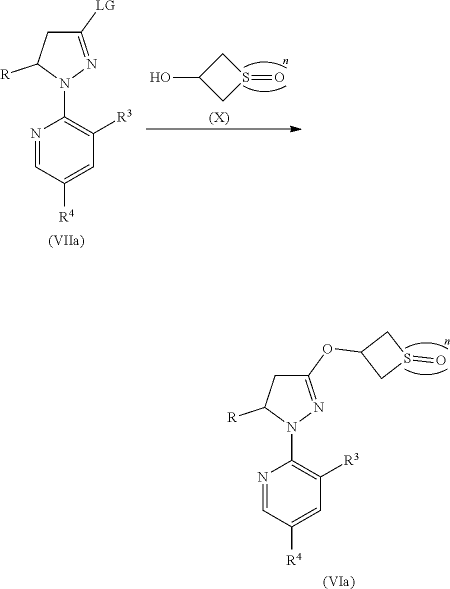
wherein, n=0 to 2; R³, R⁴, R and LG are as defined herein above;
c) converting the compound of formula (VIa) to a compound of formula (Va), in the presence of a suitable oxidizing agent and a suitable solvent 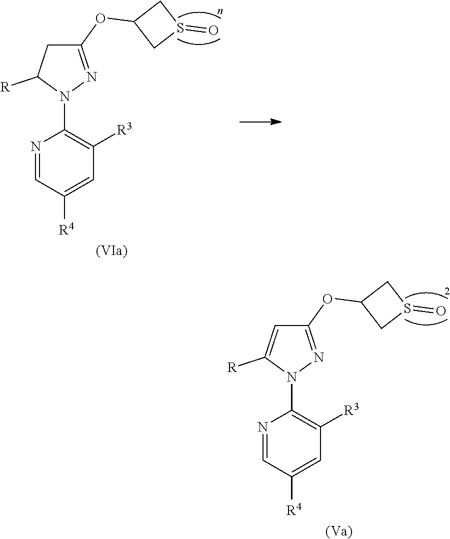
wherein, n=0 to 2; R, R³ and R⁴ are as defined herein above;
d) converting the compound of formula (Va) to a compound of formula (IVa) 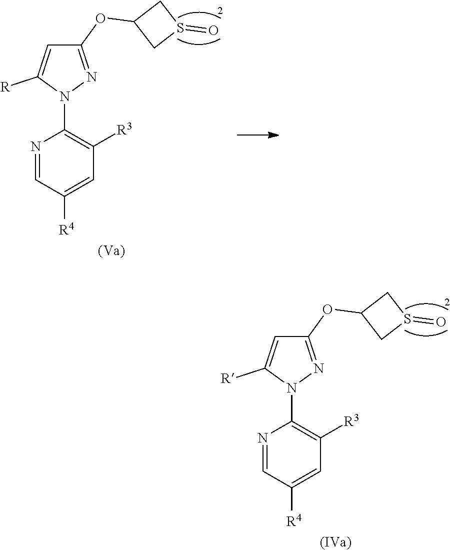
wherein R′ represents COOH or COX; X, R³, R⁴ and R are as defined herein above;
e) reacting the compound of formula (IVa) with a compound of formula (III-1) to obtain a compound of formula (IIa); 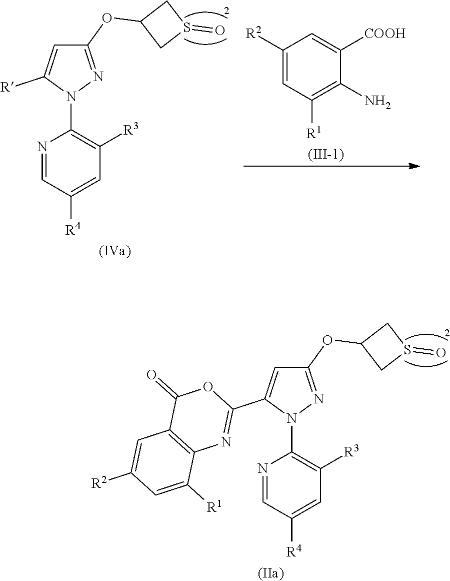
wherein, R′, R¹, R², R³ and R⁴ are as defined herein above;
or
reacting a compound of formula (IVa) with a compound of formula (IIIa-1) to obtain a compound of formula (I); 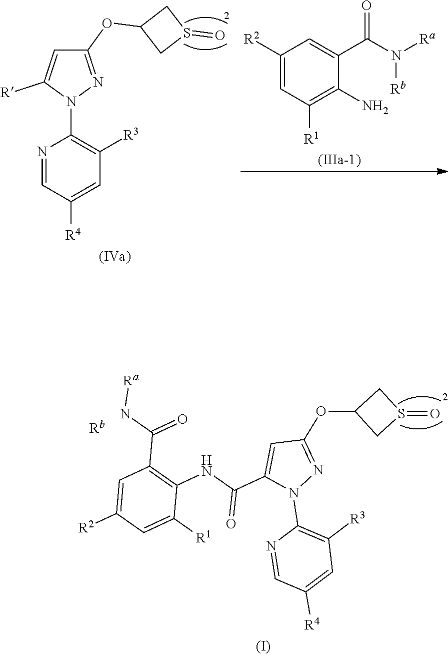
wherein, R′, R^{a}, R^{b}, R¹, R², R³ and R⁴ are as defined herein above.
f) reacting a compound of formula (IIa) with an amine (R^{a}R^{b}NH) to obtain a compound of formula (Ia) 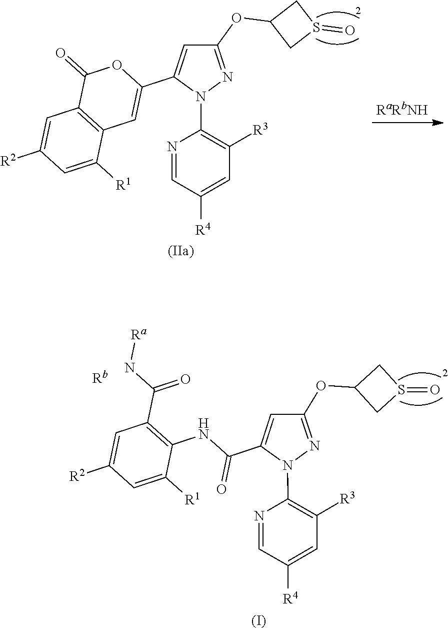
wherein, R^{a}, R^{b}, R¹, R², R³ and R⁴ are as defined herein above.

8. The process as claimed in claim 1, wherein said leaving group (LG) is selected from the group consisting of halogen, OMs, OTf and OTs.

9. The process as claimed in claim 7, wherein said leaving group (LG) is selected from Cl, Br or OMs.

10. The process as claimed in claim 1, wherein said step (a) and step (d-2) is carried out in the presence of suitable halogenating agent.

11. The process as claimed in claim 3, wherein said suitable halogenating agent is selected from the group consisting of phosphoryl chloride, phosphoryl bromide, phosphorus trichloride, phosphorus pentachloride, methanesulfonyl chloride, tosyl chloride, bromine, chlorine, thionyl chloride, oxalyl chloride, CX₄—P(Ph)₃, phosgene and cyanuric chloride.

12. The process as claimed in claim 1, wherein the oxidizing agent is selected from the group consisting of manganese dioxide (MnO₂), potassium permanganate (KMnO₄), nitric acid (HNO₃), sodium nitrite (NaNO₃), activated charcoal, palladium on carbon, copper(I) chloride, copper(II) chloride, iron(III) chloride (FeCl₃), copper(II) acetate, oxygen, hydrogen peroxide, tertiary butyl hydrogen peroxide (TBHP), sulfuric acid, oxone, H₂O₂—AcOH, V₂O₅—H₂O₂ selenioum dioxide, selenous acid, and CuCl—AcOH and mixture thereof.

13. The process as claimed in claim 1, wherein the oxidizing agent is selected from the group consisting of nitric acid (HNO₃), H₂O₂—AcOH, potassium permanganate and mixture thereof.

14. The process as claimed in claim 1, wherein the step (b) is carried out in the presence of a suitable catalyst is selected from the group consisting of copper chloride, copper iodide, trisodium or tripotassium phosphate, tetramethylethylenediamine, ethylenediamine, ferric chloride and mixture thereof.

15. The process as claimed in claim 1, wherein said step (b) is carried out in the presence of a suitable base.

16. The process as claimed in claim 3, wherein the suitable base is an inorganic base selected from the group consisting of lithium carbonate (Li₂CO₃), potassium carbonate (K₂CO₃), lithium hydroxide (LiOH), cesium carbonate (Cs₂CO₃), sodium hydroxide (NaOH), potassium hydroxide (KOH), cesium hydroxide (CsOH), calcium hydroxide (Ca(OH)₂), sodium diphosphate (Na₂HPO₄), sodium phosphate (Na₃PO₄), potassium diphosphate (K₂HPO₄), potassium phosphate (K₃PO₄), sodium methoxide (NaOCH₃) and mixture thereof.

17. The process as claimed in claim 3, wherein the suitable base is an organic base selected from the group consisting of ethylamine, triethylamine, isopropylamine diisopropylamine, triisopropylamine, pyridine, piperidine, methylmorpholine, N-methylpiperidine N,N-(dimethylamino)pyridine (DMAP), lutidine, collidine, tetramethylammonium hydroxide, tetrabutylammonium hydroxide, choline hydroxide; amidine base for example, 1,5,7-triazabicyclo[4.4.0]dec-5-ene, (TBD), 2,3,4,6,7,8,9,10-octahydropyrimidol[1,2-a]azepine (DBU) 1,5-diazabicyclo[4.3.0]non-5-ene (DBN), 1,4-diazabicyclo[2.2.2]octane (DABCO, triethylenediamine) and mixture thereof.

18. The process as claimed in claim 1, wherein the said process is carried out in the presence of a suitable solvent.

19. The process as claimed in claim 18, wherein the suitable solvent is selected from the group consisting of aliphatic, alicyclic or aromatic hydrocarbons, halogenated hydrocarbons, ethers, nitriles, amides, alcohols, water and combinations thereof.

20. The process as claimed in claim 1, wherein the suitable hydrolyzing agent used in the hydrolysis step is acid.

21. The process as claimed in claim 20, wherein said hydrolyzing agent is selected from group consisting of aqueous H₂SO₄, perchloric acid, HCl or a mixture thereof.

22. The process as claimed in claim 1, wherein the said process is carried out in the presence of a suitable catalyst.

23. The process as claimed in claim 22, wherein said suitable catalyst is selected from group consisting of sodium tungstate, tungstic acid, trifluroacetic acid, acetic acid, selenioum dioxide, selenous acid, vanadium pentoxide (V₂O₅).

24. A compound of formula (Z) or salts thereof, 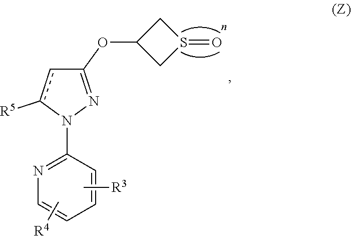
wherein,
R³ is selected from the group consisting of halogen, C₁-C₄ alkyl and C₁-C₄ haloalkyl;
R⁴ is selected from the group consisting of hydrogen, halogen, C₁-C₄ alkyl and C₁-C₄ haloalkyl;
R⁵ is selected from the group consisting of CX₃, CN, COOH, COX or COOR^{c};
R^{c} represents C₁-C₄ alkyl; X represents halogen;
n represents an integer selected from 1 to 2; and [symbol]
represents single or double bond.

25. The process as claimed in claim 1,
wherein,
n is 2;
R′ is COX or COOH;
R³ is halogen; and
R⁴ is hydrogen.

26. The process as claimed in claim 2,
wherein,
n is 2;
R′ is selected from the group consisting of COX or COOH;
R^{a} and R^{b} are independently selected from the group consisting of hydrogen, C₁-C₆ alkyl, and C₃-C₆ cycloalkyl-C₁-C₄ alkyl;
R¹ and R² are independently selected from the group consisting of hydrogen, halogen, cyano, C₁-C₆ alkyl;
R³ is halogen; and
R⁴ is hydrogen.
